(19) United States
(12) Patent Application Publication
(10) Pub. No.: US 2022/0007607 A1
(43) Pub. Date: Jan. 13, 2022

(54) HIGH THROUGHPUT PROTOPLAST ISOLATION AND TRANSFORMATION OF PLANT CELLS FROM A NOVEL LEAF-BASED CELL CULTURE-DERIVED SYSTEM

(71) Applicant: UNIVERSITY OF TENNESSEE RESEARCH FOUNDATION, Knoxville, TN (US)

(72) Inventors: Mst Shamira SULTANA, Knoxville, TN (US); Scott Christopher LENAGHAN, Knoxville, TN (US); C. Neal STEWART, Knoxville, TN (US); Taylor FRAZIER-DOUGLAS, Knoxville, TN (US)

(21) Appl. No.: 17/309,170

(22) PCT Filed: Nov. 5, 2019
(86) PCT No.: PCT/US2019/059792

§ 371 Date: May 3, 2021

Related U.S. Application Data
(60) Provisional application No. 62/807,907, filed on Feb. 20, 2019.
(60) Provisional application No. 62/755,642, filed on Nov. 5, 2018.

(51) Int. Cl. A01H 4/00

(52) U.S. Cl. CPC A01H 4/005; A01H 4/001

(57) ABSTRACT

The present disclosure describes novel methods for preparing leaf-derived plant cell suspension cultures. The cell suspension cultures produced by the methods provide a renewable and efficient source of protoplasts for high-throughput transformation and other uses. Applicants have surprisingly found that protoplasts can be obtained from the cell suspension cultures with inexpensive cell wall degrading enzymes and that the protoplasts provide increased transformation efficiencies relative to protoplasts from other sources.

### CROSS-REFERENCE TO RELATED APPLICATIONS

This application claims priority to provisional applications U.S. Ser. No. 62/755,642 filed Nov. 5, 2018 and U.S. Ser. No. 62/807,907 filed Feb. 20, 2019, which are incorporated herein by reference in their entireties.

### SEQUENCE LISTING

The instant application contains a Sequence Listing which has been submitted in ASCII format via EFS-Web and is hereby incorporated by reference in its entirety. Said ASCII copy, created on Nov. 4, 2019 is named SULTANA_P13090WO00_SEQ_LISTING_ST25.txt and is 5,529 bytes in size.

### FIELD OF THE INVENTION

This invention relates to the field of plant biotechnology. In particular this invention relates to methods for preparing plant cell suspension cultures as well as the use of such cell suspension cultures as a renewable and efficient source of protoplasts for transformation and high throughput transient assays.

### BACKGROUND OF THE INVENTION

Soybean is an important oilseed crop that is grown for food, animal feed, biofuel, and other industrial uses, in terms of worldwide cultivation of genetically engineered varieties, soybean occupies the highest land area of all crop species. While soybean transformation is genotype dependent with regards to efficiency and ease, much effort has been expended to enable reverse genetic studies in the species, but it lags behind many other crops. Several transformation methodologies have been developed including those using immature embryos and mature seeds as explants for introduction of transgenes by Agrobacterium tumelaciens and biolistics.

Given the level of effort and time required for stable soybean transformation, other easy-to-transform dicot species such as Nicotiana benthamiana, Arabidopsis and Nicotiana tabacum have been employed as proxies to estimate transgene expression and utility in soybean leaves. While these proxies are considered to be suboptimal-to-minimally relevant to soybean as a crop, hairy root production via Agrobacterium rhizogenes is routinely performed in functional genomics studies of soybean; it is, however, labor-intensive and only relevant to root traits.

Soybean genomics, biotechnology and synthetic biology would benefit from the development of a facile system for high-throughput analysis of genetic elements; of both endogenous and synthetic origins. Plant protoplasts have long been considered as species-relevant proxies for screening of DNA sequence function relative to cell wall synthesis, gene expression; and signal transduction. In some cases, protoplast data may be relevant to their donor organs and tissues under various environmental constraints, requiring empirical testing to validate tissue-specific function of regulatory elements such as promoters; enhancers and transcription factors.

As can be seen a need exists in the art for cell culture systems suitable for protoplast isolation and high-throughput transformation in soybean and other plant species.

### BRIEF SUMMARY OF THE INVENTION

The methods of the invention provide plant cell suspension cultures as a source of protoplasts for high-throughput transformation and other uses. A preferred embodiment includes methods of producing plant cell suspension cultures. The methods comprise generating callus from leaf tissue, transferring the callus to a liquid media to form a suspension of cells, subculturing the suspension of cells under conditions sufficient to maintain the cells in a viable state, and filtering the suspension of cells to remove large cell clusters.

In some embodiments, generating callus from leaf tissue comprises placing the leaf tissue adaxial-side down in callus induction media and maintaining the leaf tissue under light for a time sufficient to generate callus, preferably less than about three weeks. In some embodiments, the callus induction media is supplemented with an auxin. Preferably, the auxin is 2,4-dichlorophenoxyacetic acid. Preferably, the callus induction media comprises from about 5 μl to about 40 μM 2,4-dichlorophenoxyacetic acid. In some embodiments, the callus is divided into pieces prior to transferring the callus to the liquid media. Preferably, the suspension of cells is maintained in the dark. In some embodiments, the methods include collecting supernatant from the suspension of cells after allowing the suspension of cells to settle for a period of time, preferably after settling for about 30 minutes. Preferably, the filtering removes cell clusters larger than about 100 μm. In some embodiments, the methods comprise cryopreserving the cell suspension cultures. In some embodiments, the plant is selected from soybean, potato, tomato, bean, pea, sunflower, maize, rice, barley, and wheat. Preferably, the plant is soybean.

In some embodiments, the methods comprise obtaining protoplasts from the cell suspension cultures. In some embodiments, the methods comprise introducing a nucleic acid into the protoplast. Preferably, the nucleic acid comprises a promoter. Preferably, the nucleic acid comprises a reporter gene. In some embodiments, the introducing is by microinjection, electroporation, Agrobacterium-mediated transformation, polyethylene glycol (PEG)-mediated transformation, or microprojectile bombardment. Preferably, the introducing is by PEG-mediated transformation. In some embodiments, introducing the nucleic acid is automated.

A preferred embodiment includes methods of obtaining protoplasts from a plant. The methods comprise providing leaf tissue from the plant, placing the leaf tissue adaxial-side down in callus induction media, incubating under light for a time sufficient to generate callus, dividing the callus into pieces, transferring the callus pieces to a liquid media to form a suspension of cells, subculturing the suspension of cells under conditions sufficient to maintain the cells in a viable state, filtering the suspension of cells to remove large cell clusters, recovering the cells from the liquid media, and removing the cell wall from the cells with suitable enzymes to form protoplasts.

A preferred embodiment includes plant cell suspension cultures and plant protoplasts prepared by the foregoing methods.

While multiple embodiments are disclosed, still other embodiments of the present invention will become apparent to those skilled in the art from the following detailed description, which shows and describes illustrative embodiments of the invention. Accordingly, the figures and detailed description are to be regarded as illustrative in nature and not restrictive.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1 shows cell culture development from soybean leaves. The photographs show in vitro grown plants, 3-wk-old leaves used for callus induction, 3 wk of incubation at 24 C under white light induced callus production, 4 wk of incubation at 25 C in the dark proliferated callus, and fine cell cultures Obtained from callus in 5 wk.

FIGS. 2A-C show changes in callus color after light exposure. FIG. 2A shows callus maintained in the dark at 25° C., FIG. 2B shows callus changed colors from pale/white to light green within 3 wk under white light, and FIG. 2C shows callus changed colors from light to darker green within 3 more wk.

FIGS. 3A-B show age of culture affects cell proliferation. FIG. 3A shows cell clusters from 6-mo-old of culture, which had reduced active cell proliferation. FIG. 3B shows healthy and vigorous proliferation of cells 7 d after subculture.

FIG. 4 shows the maintenance of soybean cell suspension culture. The photographs show 7-day-old culture, 5 mL cells of 7-day-old cultures mixed with 45 mL fresh media for replenishing cultures, and cells incubating at 24° C. in the dark on a rotary shaker at 80 rpm.

FIGS. 5A-B show rearrangement of cell shapes 4 d after subculture. Figure SA shows a clump of round/oval shape cells and FIG. 5B shows an elongated cell.

FIGS. 6A-B show that cell shape influence distribution patterns. FIG. 6A shows round-to-oval cells tended to clump together and FIG. 6B shows elongated cells remained more widely distributed.

FIGS. 7A-F show that the proportion of elongated cells increases with time after subculture. FIG. 7A shows day 0, FIG. 7B shows day 2, FIG. 7C shows day 4, FIG. 7D shows day 6, FIG. 7E shows day 8, and FIG. 7F shows day 10.

FIGS. 8A-B show the effects of sub-culturing on cell culture growth. FIG. 8A shows fresh weight over time after sub-culturing, a plate reader was used to measure optical density over time after sub-culturing, error bars represent standard error (n=6). Same letters above error bar indicate no significant difference according to the ANOVA Fisher's test (p<0.05), FIG. 8B shows photographs of harvested cells on filter paper.

FIG. 9 shows the stages of tissue harvesting for protoplast isolation. The photographs show 5 mL PCV collected from 4 d after subculture, 30-d-old immature cotyledons harvested from greenhouse grown plants, 15-d-old stems grown in soil and harvested, and 17-d-old leaves grown in soil (10 d in light+7 d in dark).

FIGS. 10A-C show the effects of age and type of tissue on protoplast production and viability. FIG. 10A shows the age of cell culture after sub-culture on protoplast yield. FIG. 10B shows the effect of time after sub-culture on protoplasts viability. FIG. 10C shows that recovery of millions of protoplasts/g and viability were affected by the source of tissue. Error bars represent standard error (n=6). Same letters above bars indicate no significant difference according to the ANOVA Fisher's LSD test (p<0.05).

FIGS. 11A-C show the vectors used in protoplast transformation experiments. FIG. 11A shows the test vector structure used an internal standard an orange fluorescent protein reporter CaMV 35S promoter::TagRFP-T cassette and the green fluorescent protein reporter test promoter::mEmerald cassette. The line represents test promoters with promoter length (base pairs; bp) being indicated above each line. NosT; Nos terminator. FIG. 11B shows the positive control used identical CaMV 35S promoters driving both reporter genes. FIG. 11C shows the negative control vector contains a promoterless mEmerald cassette. All plasmids are binary vectors and harbor a kanamycin resistant gene for the bacterial selection.

FIG. 12 shows optimization of soybean protoplast transformation efficiency by tissue source, duration of incubation based on polyethylene glycol (PEG). Protoplasts were transfected with 10 μg of the plasmid (pB2GW7: 9983 bp) DNA. Error bars represent standard error (n=6). Same letters above bars indicate no significant difference according to the ANOVA Fisher's LSD test (p<0.05).

FIGS. 13A-D show transient expression in protoplasts. Bright-field images, MT fluorescence. GFP fluorescence and merged of two-color fluorescence were shown for each tissue source. Fluorescence was assayed at 48 hr. after transformation using confocal microscopy. The expression was observed in cell culture, leaf, stem, and immature cotyledon-derived protoplasts. FIG. 13A shows cell culture, FIG. 13B shows leaf FIG. 13C shows stem, and FIG. 13D shows immature cotyledon. The scale bar ti represents 100 μm and low-magnification (10×) image of protoplasts.

FIGS. 14A-D show quantitative measurement of promoter's strength in different tissues-derived protoplasts and association analysis. Fluorescence was quantified 48 h after transfection. Note that the positive control vector includes 35S: OFP and 35S: GFP as a reference expression and promoter strength was determined by ratioed GFP fluorescence against OFP fluorescence. Positive control vector 35S: 35S promoters were standardized to 1.0, which was used to normalize the strength of promoters of interest. The relative promoter's strength was shown in soybean cell cultures, leaves, stems, and immature cotyledons-derived protoplasts. FIG. 14A shows cell culture, FIG. 14B shows leaves, FIG. 14C shows stems, and FIG. 14D shows immature cotyledons. Error bars represent standard error (n=6). Same letters above bars indicate no significant difference according to the ANOVA Fisher's LSD test (p<0.05). FIG. 14E shows linear regression analysis of relative promoter's strength on leaf and cell culture-derived protoplasts, stem and cell culture-derived protoplasts, and immature cotyledon and cell culture-derived protoplasts.

FIGS. 15A-D show qRT-PCR transcript abundance of target promoter gene from left in soybean tissues. Transcription of various genes were estimated in cell culture, leaves, stems and immature cotyledons. FIG. 15A shows cell cultures, FIG. 15B shows leaves, FIG. 15C shows stems, and FIG. 15D shows immature cotyledons. The relative levels of transcript were normalized to soybean ubiquitin (GmUBI3) and actin11 gene. Error bars represent standard error (n=3). Same letters above bars indicate no significant difference according to the ANOVA Fisher's LSD test (p<0.05).

FIG. 16 shows the relative expression of endogenous target promoter gene across tissue types. Ubiquitin, tubulin, Hsp90, ribosomal protein, GAL and actin transcripts abundance in leaf, cell suspension culture, stem and immature cotyledon. The relative levels of transcripts were normalized to soybean actin11 gene. Error bars represent standard error (n=6). Same letters above bars indicate no significant difference according to the ANOVA Fisher's LSD test (p<0.05).

FIG. 17 shows protoplast expression using an automated transformation system. Protoplasts were transfected with a pB2GW7 construct fused to enhanced 35S promoter-mEmerald and pMTV construct fused to ubiquitin promoter-GFP and 35S promoter-OFP. After overnight incubation, expression of reporter gene was visualized using a confocal microscope. The scale bar represents 100 μm and low-magnification (10×) image of protoplasts.

### DETAILED DESCRIPTION OF THE INVENTION

So that the present invention may be more readily understood, certain terms are first defined. Unless defined otherwise, all technical and scientific terms used herein have the same meaning as commonly understood by one of ordinary skill in the art to which embodiments of the invention pertain. Unless otherwise defined, all technical and scientific terms used herein have the same meaning as commonly understood by one of ordinary skill in the art. Generally, the definitions of various terms used herein are well known and conventionally used in the art.

The singular terms “a”, “an”, and “the” include plural referents unless context clearly indicates otherwise. Similarly, the word “or” is intended to include “and” unless the context clearly indicate otherwise. The word “or” means any one member of a particular list and also includes any combination of members of that list.

Numeric ranges recited within the specification are inclusive of the numbers defining the range and include each integer within the defined range. Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges, fractions, and individual numerical values within that range. For example, description of a range such as from 1 to 6 should be considered to have specifically 5 disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4%. This applies regardless of the breadth of the range.

The term “about,” as used herein, refers to variation in the numerical quantity that can occur, for example, through typical measuring techniques and equipment, with respect to any quantifiable variable, including, but not limited to, mass, volume, time, and concentration. Further, given solid and liquid handling procedures used in the real world, there is certain inadvertent error and variation that is likely through differences in the manufacture, source, or purity of the ingredients used to make the compositions or carry out the methods and the like. The term “about” also encompasses amounts that differ due to different equilibrium conditions for a composition resulting from a particular initial mixture. The term “about” also encompasses these variations. Whether or not modified by the term “about,” the claims include equivalents to the quantities.

As used herein, the term “suspension cell” or “suspension culture” (for example; “plant suspension cell line”, “plant suspension culture”, “plant suspension cell(s)” or “plant cell suspension culture”) are a specialized population of homogenous, undifferentiated plant cells grown in liquid nutrient or culture media. The cells are typically suspended within the media as opposed to adhering to a surface.

As used herein, the term “suspending” as used herein is intended to include any placement of a solid (e.g., plant cells) in a liquid whether or not an actual suspension is created. As such, the term “suspending” is intended to include any mixing of a solid in a liquid or any other placement of a solid in a liquid. As a result; the term “suspension” is likewise not intended to be limited to suspensions, but rather is intended to mean any mass having a solid present in a liquid.

As used herein, the term “calli” or “callus” refers to a group of structurally undifferentiated cells derived from any plant parts of plants. Once a plant has been induced to form callus, the callus tissue can be used as an experimental system to investigate and solve a broad range of basic research problems, and to introduce foreign genes into a variety of horticultural and agronomic plants for the purpose of crop improvement.

As used herein, the term “plant tissue” refer to an organized grouping of differentiated and undifferentiated plant cells. For example, a plant tissue would include plant embryos, leaves, pollen, roots, root tips, anther, silks, flowers, kernels, bulbs, tubers, rhizomes, and seeds. Further; plant tissue includes “plant organs” that constitute plant tissue or a group of tissues that constitute a morphologically and functionally distinct part of a plant. Plant tissues may be located and isolated from a plant or in a plant organ, tissue or cell culture.

“Tissue culture” is a term used to describe the process where plant cells are grown outside of an intact plant in a suitable nutrient media (e.g., cell suspension media). Tissue culture is defined as a method wherein parts of a plant are transferred into an artificial environment in which they can continue to survive. The term tissue culture as understood in the art refers to cultured tissue which may consist of individual or groups of plant cells, protoplasts or whole or parts of a plant organ.

In tissue culture, plant cells can be grown on a solid surface as pale colored lumps known as callus culture or as individual or small clusters of cells known as suspension culture. Cells grown in culture are actively dividing and can be maintained indefinitely in an undifferentiated state by transferring the cells to fresh cell suspension media (subculturing or passaging). Cultured cells may also be induced to re-differentiate into whole plants. Plant tissue cultures can be initiated from almost any part of the source plant (termed explant) although younger parts of the plant are generally more useful as they contain more actively dividing cells.

The current invention provides methods for producing plant cell suspension cultures for protoplast isolation and high-throughput transformation. The methods comprise generating callus from plant tissue, transferring the callus to a liquid media to form a suspension of cells, subculturing the suspension of cells under conditions sufficient to maintain the cells in a viable state, and filtering the suspension of cells to remove large cell clusters. The cell suspension cultures produced by the methods provide a renewable source of protoplasts for transformation. The current invention further provides methods of obtaining protoplasts from a plant or from the plant cell suspension culture.

The methods comprise generating callus from a plant tissue, preferably leaf tissue. In some embodiments, the leaf pieces are placed with adaxial-side down in callus induction media. In some embodiments, the callus induction media comprises MS basal salts. “MS basal salts” is known in the art and was originally described by Murashige and Skoog, Physiology Plantarum, 15:473-497 (1962). In the methods and media of the present invention, “MS basal media” or “MS media” or “MS basal salts” as used herein includes MS basal media as described by Murashige and Skoog as well as equivalents of MS basal media. One skilled in the art would understand that equivalents of MS basal media include media that is substantially similar in contents and concentrations of salts, chemicals, etc., such that a plant, a plant tissue, or a plant cell culture would develop/grow in the same manner when exposed to MS basal media.

In some embodiments, the callus induction media is supplemented with an auxin, “Auxins” include, but are not limited to, naturally occurring and synthetic auxins. Naturally occurring auxin is indole acetic acid (“IAA”), which is synthesized from tryptophan. An exemplary synthetic auxin in dichlorophenoxyacetic acid (“2,4-D”). Other auxins include, but are not limited to, 4-chlorophenoxyacetic acid (“4-CPA”), 4-(2,4-dichlorophenoxy)butyric acid (“2,4-DB”), tris[2-(2,4-dichlorophenoxy)ethyl]phosphite (“2,4-DEP”), 2-(2,4-Dichlorophenoxy)propionic acid (“dicloroprop”), (RS)-2-(2,4,5-trichlorophenoxy)propionic acid (“fenoprop”), naphthaleneacetamide, α-naphthaleneacetic acid (“NAA”), 1-naphthol, naphthoxyacetic acid, potassium naphethenate, (2,4,5-trichlorophenoxy)acetic acid (“2,4,5-T”), indole-3-acetic acid, indole-3-butyric acid (“IBA”), 4-amino-3,5,6-trichloropyridine-2-carboxylic acid (“picloram”), 3,6-dichloro-o-anisic acid (“dicamba”), indole-3-proionic acid (“IPA”), phenyl acetic acid (“PAA”), benzofuran-3-acetic acid (“EWA”), and phenyl butric acid (“PBA”).

In some embodiments, the callus induction media comprises from about 5 μM to about 40 μM 2,4-dichlorophenoxyacetic acid. In some embodiments, the callus induction media is supplemented with from about 10 μM to about 14 μM 2,4-dichlorophenoxyacetic acid, preferably from about 11 μM to about 13 μM 2,4-dichlorophenoxyacetic acid. Most preferably, the callus induction media is supplemented with about 12 μM 2,4-dichlorophenoxyacetic acid. Less than about 12 μM 2,4-dichlorophenoxyacetic acid may produce lesser amounts of callus while higher than about 12 μM can result in brownish leaf edges. An exemplary callus induction media comprises MS basal salts, 3% sucrose, 150 mg/L casein hydrolysate, 0.8% agar supplemented with 12 μM 2,4-dichlorophenoxyacetic acid, pH 5.7.

In the initial stage of callus induction, the culture is maintained under light for a time sufficient to generate callus. Light incubation is needed for inducing callus from leaf tissue because leaf pieces can gradually turn a black color under dark incubation. In some embodiments, the growth conditions suitable for initiating callus include 24° C. under white light with a 16 h day/8 h night photoperiod. Preferably, the time sufficient to generate callus is less than about three weeks because induced callus can turn into a black color when incubated under light longer than 3 weeks. After about 3 weeks of culture, callus is excised from the leaf pieces and plated on fresh callus induction media for proliferation. Once established, callus proliferation occurs under dark conditions to obtain friable and soft callus. Callus proliferation under light turns a brownish color with hard morphology and is not suitable for producing cell suspension cultures.

The methods comprise initiating a cell suspension from the callus. In some embodiments, the methods comprise transferring the callus to a liquid media to form a suspension of cells. In some embodiments, the liquid media comprises MS basal salts. In some embodiments, the liquid media is supplemented with an auxin. Preferably, the auxin is 2,4-dichlorophenoxyacetic acid. In some embodiments, the liquid media is supplemented with about 0.92 μM 2,4-dichlorophenoxyacetic acid. An exemplary liquid media comprises MS basal salts. 3% sucrose, 0.92 μM 2,4-dichlorophenoxyacetic acid, pH 5.6.

In some embodiments, the callus is gently divided into pieces immediately prior to transferring the callus to the liquid media. As used herein, the term “divide” and any other word forms or cognates thereof, such as, without limitation, “dividing”, includes the process of reducing a composition from a first size to a smaller size by any suitable method or process, including, without limitation, chopping, cutting, dicing, mincing; or slicing.

The methods comprise subculturing the suspension of cells under conditions sufficient to maintain the cells in a viable state. The term “subculturing” refers to conditions that typically involve harvesting (withdrawing) cells, diluting or splitting the cells with fresh cell suspension media; and cultivating the diluted or split cell culture. Preferably, the suspension of cells is maintained in the dark with shaking. In some embodiments, the methods comprise collecting supernatant from the suspension of cells after allowing the suspension of cells to settle for a period of time and transferring the supernatant to fresh media. Preferably the period of time is at least about 10 minutes, more preferably from about 20 minutes to about 40 minutes, or most preferably about 30 minutes.

In some embodiments, the methods comprise filtering the suspension of cells to remove large cell clusters. The term “filtering” as used herein is synonymous with sieving, straining, and the like. In particular embodiments, a size-exclusion screen or mesh is used to isolate the cells from the cell clusters having a larger diameter. In some embodiments, the cell clusters are those greater than about 50 μm, about 75 μm, about 100 μm, about 125 μm, or about 175 μm in diameter. Preferably, the cell clusters are those greater than about 100 μm. In some embodiments, the filtering removes the cell clusters having a diameter larger than about 50 μm, about 75 μm, about 100 μm, about 125 μm, or about 175 μm. Preferably, the filtering removes the cell clusters having a diameter larger than about 100 μm.

In some embodiments, the methods comprise recovering a plant cell from the liquid media. As used herein, the term “recovering” refers to the isolation of the plant cells or other biological materials such that it is significantly free of any media component.

The methods comprise obtaining protoplasts from the cell suspension culture. The term “protoplast”, as used herein, refers to a plant cell that had its cell wall completely or partially removed, with the lipid bilayer membrane thereof naked. Protoplasts are preferably produced by subjecting plant cells to enzymatic breakdown of the cell walls. Cell wall degrading enzymes include cellulases, hemicellulases, ligninases, and pectinases. In a preferred embodiment, the cell wall degrading enzymes are food-grade enzymes. It is an advantage of the present methods that low-cost, food-grade enzymes are effective in obtaining plant protoplasts from the leaf-derived plant cell suspension cultures. In contrast, such food-grade enzymes are ineffective at digestion and fail to yield viable protoplasts when leaf tissue is used directly.

The methods described herein include introducing a nucleic acid into a plant protoplast. As used herein “introducing” is intended to mean presenting to the plant the nucleotide construct in such a manner that the construct gains access to the interior of the cell. The methods herein do not depend on a particular method for introducing a nucleotide construct to a plant protoplast, only that the nucleic acid gains access to the interior of at least one protoplast. Methods for introducing nucleic acids into plants are known in the art including, but not limited to, stable transformation methods, transient transformation methods, microinjection, and virus-mediated methods. A “stable transformation” is one in which the nucleotide construct introduced into a plant integrates into the genome of the plant and is capable of being inherited by progeny thereof. “Transient transformation” means that a nucleotide construct introduced into a plant does not integrate into the genome of the plant. The nucleotide constructs of the embodiments may be introduced into plants by contacting plants with a virus or viral nucleic acids. Generally, such methods involve incorporating a nucleotide construct within a viral DNA or RNA molecule. Methods for introducing nucleotide constructs into plants and expressing a protein encoded therein, involving viral DNA or RNA molecules, are known in the art. See, for example, U.S. Pat. Nos. 5,889,191, 5,889,190, 5,866,785, 5,589,367, and 5,316,931).

Transformation protocols as well as protocols for introducing nucleotide sequences ti into plants may vary depending on the size of the nucleic acid molecule and the number of protoplasts available. Suitable methods of introducing nucleotide sequences into plant cells and subsequent insertion into the plant genome include microinjection (Crossway et al. (1986) Biotechniques 4:320-334), electroporation (Riggs et al. (1986) Proc. Natl. Acad. Sci. USA 83:5602-5606), Agrobacterium-mediated transformation (U.S. Pat. Nos. 5,981,840 and 5,563,055), direct gene transfer (Paszkowski et al. (1984) EMBO J. 3:2717-2722), and ballistic particle acceleration (see, for example, U.S. Pat. Nos. 4,945,050; 5,879,918; 5,886,244; 5,932,782; Tomes et al. (1995) In Plant Cell, Tissue, and Organ Culture: Fundamental Methods, ed. Gamborg and Phillips (Springer-Verlag, Berlin); and McCabe et al. (1988) Biotechnology 6:923-926). In some embodiments, the introducing is by polyethylene glycol (PEG)-mediated transformation.

In some embodiments, the methods of the current invention provide increased protoplast transformation efficiency relative to other sources of protoplasts. For example, in certain embodiments, the methods provide about 13% transformation efficiency using leaf protoplasts compared to about 30% transformation efficiency using leaf-derived cell suspension culture protoplasts.

The steps in the methods of the present invention may be performed manually or by automation. For high-throughput transient expression, it is preferable that the methods are automated. In a preferred embodiment, one or more steps are automated. One embodiment of the current invention provides high throughput automated handling for protoplast isolation and/or transformation. Use of robots and other automated devices in the laboratory is common in the art (see, for example, Dlugosz et al, 2016, J Vis Exp (115):54300). In a high throughput automated method of the current invention, a plurality of plant protoplasts are transformed with a plurality of nucleic acids to produce a plurality of transformed plant protoplasts. In some embodiments, the protoplasts produced by the methods of the current invention provide an increased protoplast transformation rate when used with a high throughput automated transformation system.

As used herein, the terms “polynucleotide”, “nucleic acid”; and “nucleic acid molecule” are used interchangeably, and may encompass a singular nucleic acid; plural nucleic acids; a nucleic acid fragment, variant, or derivative thereof, and nucleic acid construct (e.g., messenger RNA (mRNA) and plasmid DNA (pDNA)). A polynucleotide or ti nucleic acid may contain the nucleotide sequence of a full length cDNA sequence, or a fragment thereof, including untranslated 5′ and/or 3′ sequences and coding sequence(s). A polynucleotide or nucleic acid may be comprised of any polyribonucleotide or polydeoxyribonucleotide, which may include unmodified ribonucleotides or deoxyribonucleotides or modified ribonucleotides or deoxyribonucleotides. For example, a polynucleotide or nucleic acid may be comprised of single- and double-stranded DNA; DNA that is a mixture of single- and double-stranded regions; single- and double stranded RNA; and RNA that is mixture of single- and double-stranded regions. Hybrid molecules comprising DNA and RNA may be single-stranded, double-stranded, or a mixture of single- and double-stranded regions. The foregoing terms also include chemically, enzymatically, and metabolically modified forms of a polynucleotide or nucleic acid.

It is understood that a specific DNA refers also to the complement thereof, the sequence of which is determined according to the rules of deoxyribonucleotide base-pairing.

“Exogenous nucleic acid” is a nucleic acid that is not native to a specified system (e.g., a germplasm, plant, variety, etc.), with respect to sequence, genomic position, or both. As used herein, the terms “exogenous” or “heterologous” as applied to polynucleotides or polypeptides typically refers to molecules that have been artificially supplied to a biological system (e.g., a plant cell, a plant gene, a particular plant species or variety or a plant chromosome under study) and are not native to that particular biological system. The terms can indicate that the relevant material originated from a source other than a naturally occurring source, or can refer to molecules having a non-natural configuration, genetic location or arrangement of parts. In contrast, for example, a “native” or “endogenous” gene is a gene that does not contain nucleic acid elements encoded by sources other than the chromosome or other genetic element on which it is normally found in nature. An endogenous gene, transcript or polypeptide is encoded by its natural chromosomal locus, and not artificially supplied to the cell.

As used herein, the term “gene” refers to a nucleic acid that encodes a functional product (RNA or polypeptide/protein). A gene may include regulatory sequences preceding (5′ non-coding sequences) and/or following (3′ non-coding sequences) the sequence encoding the functional product.

As used herein, the term “polypeptide” includes a singular polypeptide, plural polypeptides, and fragments thereof. This term refers to a molecule comprised of monomers (amino acids) linearly linked by amide bonds (also known as peptide bonds). The term “polypeptide” refers to any chain or chains of two or more amino acids, and does not refer to a specific length or size of the product. Accordingly, peptides, dipeptides, tripeptides, oligopeptides, protein, amino acid chain, and any other term used to refer to a chain or chains of two or more amino acids, are included within the definition of “polypeptide”, and the foregoing terms are used interchangeably with “polypeptide” herein. A polypeptide may be isolated from a natural biological source or produced by recombinant technology, but a specific polypeptide is not necessarily translated from a specific nucleic acid. A polypeptide may be generated in any appropriate manner, including for example and without limitation, by chemical synthesis. Likewise, a polypeptide may be generated by expressing a native coding sequence, or portion thereof, that is introduced into an organism in a form that is different from the corresponding native coding sequence.

The term “promoter” refers to a DNA sequence capable of controlling the expression of a nucleic acid coding sequence or functional RNA. In examples, the controlled coding sequence is located 3′ to a promoter sequence. A promoter may be derived in its entirety from a native gene, a promoter may be comprised of different elements derived from different promoters found in nature, or a promoter may even comprise rationally designed DNA segments. It is understood by those skilled in the art that different promoters can direct the expression of a gene in different cell types, or at different stages of development, or in response to different environmental or physiological conditions. Examples of all of the foregoing promoters are known and used in the art to control the expression of heterologous nucleic acids. Promoters that direct the expression of a gene in most cell types at most times are commonly referred to as “constitutive promoters”. Furthermore, while those in the art have (in many cases unsuccessfully) attempted to delineate the exact boundaries of regulatory sequences, it has come to be understood that DNA fragments of different lengths may have identical promoter activity. The promoter activity of a particular nucleic acid may be assayed using techniques familiar to those in the art.

The term “operably linked” refers to an association of nucleic acid sequences on a single nucleic acid, wherein the function of one of the nucleic acid sequences is affected by, another. For example, a promoter is operably linked with a coding sequence when the promoter is capable of effecting the expression of that coding sequence (e.g., the coding sequence is under the transcriptional control of the promoter). A coding sequence may be operably linked to a regulatory sequence in a sense or antisense orientation.

The term “expression”, as used herein, may refer to the transcription and stable accumulation of sense (mRNA) or anti sense RNA derived from a DNA. Expression may also refer to translation of mRNA into a polypeptide. As used herein, the term “overexpression” refers to expression that is higher than endogenous expression of the same gene or a related gene. Thus, a heterologous gene is “overexpressed” if its expression is higher than that of a comparable endogenous gene.

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radio immunoassay, or by calorimetric, fluorogenic, chemiluminescent or other methods. Such genes include, without limitation, β-glucuronidase (GUS), luciferase (LUC), chloramphenicol fransacetylase (CAT), green fluorescent protein (GFP), and β-galactosidase. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

The plant cell suspension cultures or the plant protoplasts obtained therefrom can be optionally cryopreserved. Examples of the methods of cryopreservation are well known to a person of ordinary skill in the art and such embodiments are within the purview of the current invention. As used herein, the term “cryopreserving” or “cryopreservation” refers to the storage of biological material, e.g. cells, tissues or organs, at temperatures below 4° C. Generally, the intention of the cryopreservation is to maintain the plant cells in a preserved or dormant state, after which time the plant cells are returned to a temperature above 4° C. ti for subsequent use. Preferably, the cryopreserving temperature is below 0° C. For example, the cryopreserving temperature may be below 0° C., −5° C., −10° C., −20° C., −60° C., −80° C. or greater. Such temperatures can be reached by exposing the plant cells to liquid nitrogen, liquid helium, carbon dioxide (dry-ice′), or slurries of carbon dioxide with other solvents. In some embodiments, the cryopreserving temperature is about −20° C., about −80° C. or about −180° C.

As used herein, the term “cryopreservation agent” or “cryoprotectant” is a substance that is used to protect plant cells from freezing damage. Further, the cryopreservation agent or cryoprotectant may protect the plant cells from cold and heat shock, dehydration, and cryo-toxicity during cryopreservation. The cryopreservation agent or cryoprotectant may be cell penetrating or non-penetrating. Non-limiting examples of cryoprotectants include glycerol, DMSO (dimethyl sulfoxide), propylene glycol, ethylene glycol, acetamide, and methanol.

The methods of the current invention can be practiced in a wide variety of plants. Non-limiting examples of plants in which the current methods can be practiced include, but are not limited to, monocots and di cots such as corn (Zea mays), Brassica sp. (e.g., B. napus, B. rapa, B. juncea), particularly those Brassica species useful as sources of seed oil, alfalfa (Medicago saliva), rice (Oryza saliva), rye (Secale cereale), sorghum (Sorghum bicolor; Sorghum vulgare), millet (e.g., pearl millet (Pennisetum glaucum), proso millet (Panicum miliaceum), foxtail millet (Setaria italica), finger millet (Eleusine coracana)), sunflower (Helianthus annum), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), Sweet potato (Ipomoea batatus), cassava (Manihot esculenta), coffee (Coffea spp.), coconut (Cocos nucifera), pineapple (Ananas comosus), Citrus trees (Citrus spp.), cocoa (Theobroma cacao), tea (Camelia sinensis), banana (Musa spp.), avocado (Pervea americana), fig (Ficus casica), guava (Psidium guajava), mango (Mangifera indica), olive (Oleo europaea), papaya (Carica papaya), cashew (Anacardium occidentale), Macadamia (Macadamia integrifolia), almond (Prunus amygdalus), Sugar beets (Beta vulgaris), Sugarcane (Saccharum spp.), oats (Avena), barley (Hordeum), palm, legumes including beans and peas such as guar, locust bean, fenugreek, garden beans, cowpea, mungbean, lima bean, fava bean, lentils, chickpea, and castor, Arabidopsis, vegetables, ornamentals, grasses, conifers, crop and grain plants that provide seeds of interest, oil-seed plants, and other leguminous plants. Vegetables include tomatoes (Solanum lycopersicum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pukherrima), and chrysanthemum. Conifers include, for example, pines such as loblolly pine (Pinus taeda), slash pine (Pinus elliotil), ponderosa pine (Pinus ponderosa), lodgepole pine (Pinus contorta), Monterey pine (Pinus radiata), Douglas-fir (Pseudotsuga menziesii), Western hemlock (Tsuga canadensis), Sitka spruce (Picea glauca), redwood (Sequoia sempervirens), true firs such as silver fir (Abies amabilis) and balsam fir (Abies balsamea), and cedars such as western red cedar (Thuja plicata) and Alaska yellow-cedar (Chamaecyparis nootkatensis). In some embodiments, the plant is soybean. In an exemplary embodiment, the soybean plant is ‘Williams 82’.

### EXAMPLES

### Example 1: Initiation of Callus from Leaf Tissue and Establishment of Cell Suspension Cultures

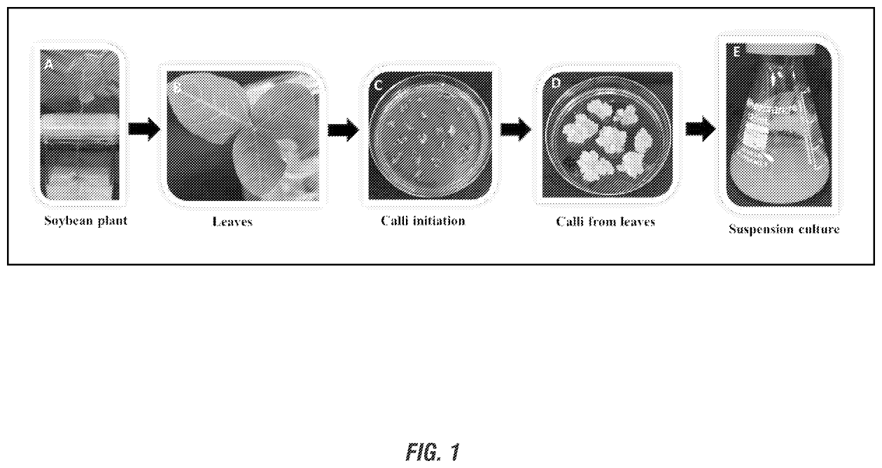

Mature ‘Williams 82’ soybean seeds collected from greenhouse-grown plants were washed with 70% ethanol for 2 min and blotted dry with filter paper. Next, the seeds were surface-sterilized in a desiccator for 12 h with chlorine gas (100 mL sodium hypochlorite (100% Clorox, commercial bleach)+3.5 mL 12 N hydrochloric acid). Sterile seeds were placed in Magenta GA-7 vessels containing germination media: Murashige and Skoog (MS) basal salts, 2% sucrose, 0.3% phytagel, pH 5.8 and placed in a growth chamber under 16 h day/8 h night cycle at 24° C. temperature for 3 wk (FIG. 1). Twenty-day-old plants were used to excise expanded leaves that were subsequently sliced into 0.5 cm-long pieces. The leaf pieces were placed with adaxial-side down in callus induction media: MS basal salts, 3% sucrose, 150 mg/L casein hydrolysate, 0.8% agar supplemented with 12 μM 2,4-dichlorophenoxyacetic acid, pH 5.7. In the initial stage, cultures were kept at 24° C. under white light and 16 h day/8 h night photoperiod for callus initiation. After 3 wk of culture, callus was excised from the leaf pieces and plated on fresh callus induction media for proliferation. Throughout the study, callus was maintained in an incubator at 25° C. in the dark. Callus was sub-cultured every 3 wk to fresh medium.

Cell suspension cultures were initiated from 1-g callus. Callus was transferred to 50 mL liquid media containing MS basal salts, 3% sucrose, 0.92 μM 2,4-dichlorophenoxyacetic acid, pH 5.6. The 50 mL cultures were maintained in 250-mL PYREX baffled Erlenmeyer flask and incubated at 24° C. in the dark on a rotary shaker at 80 rpm. To obtain fine structure cell suspension cultures, 40 mL of supernatant was removed every wk from the flasks after they had settled for 30 min and were added to 40 mL of fresh media. This procedure was continued up to 5 wk. Finally, the cell suspension cultures were filtered through a nylon mesh (100 μm) filter to remove large cell clusters. From the filtrate solution, 5 mL of fine cell suspension cultures were weekly subcultured into 45 mL of fresh MS media for subsequent perpetuation.

### Example 2: Maintenance of Cell Suspension Cultures and Growth Determination

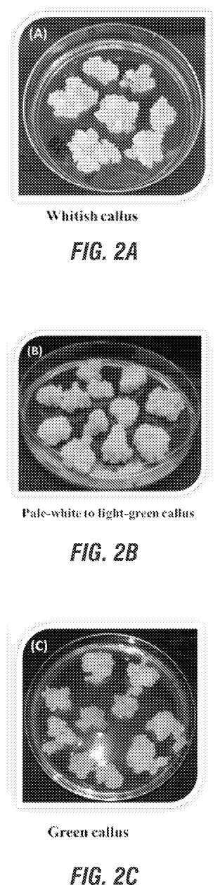

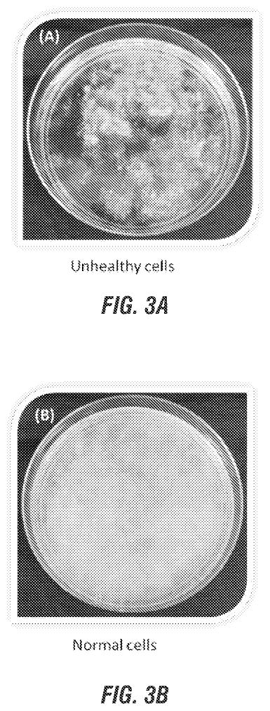

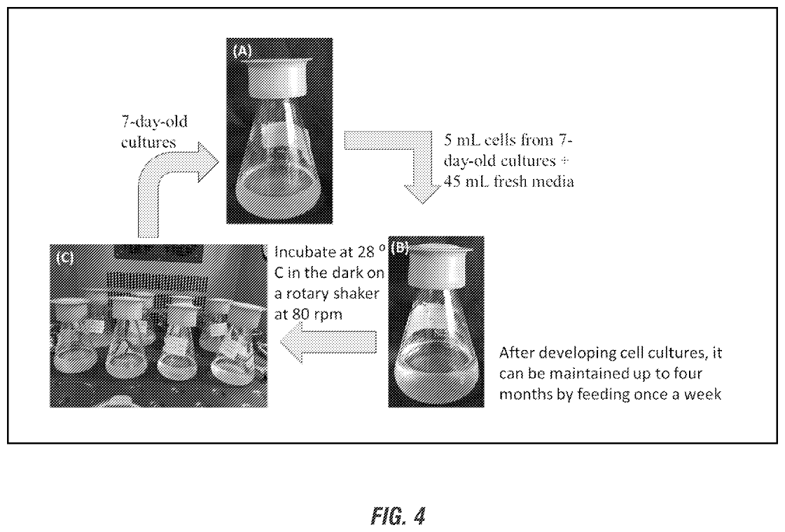

The leaf-derived callus maintained in the dark was pale (white), friable and non-embryogenic (FIG. 2A). The callus became green when exposed to white light in 6 wk (FIG. 2C). The efforts to regenerate the green callus into plants was unsuccessful. The cell suspension cultures derived from the pale-white callus was viable with no growth reductions up to 6 mo. After that, elongated cell clusters became apparent (FIG. 3), which coincided with decreased cell proliferation. The cell suspension culture was maintained by changing the media each week (FIG. 4).

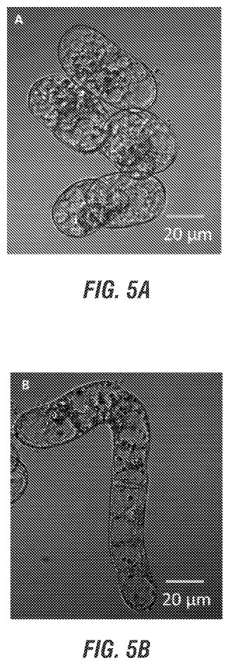

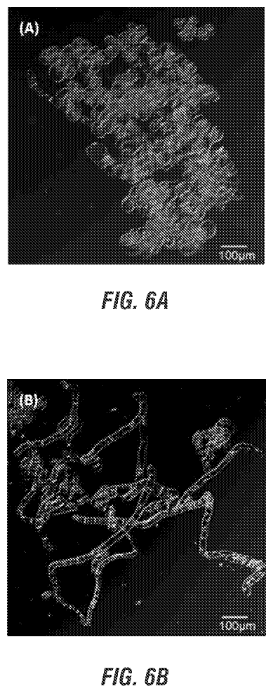

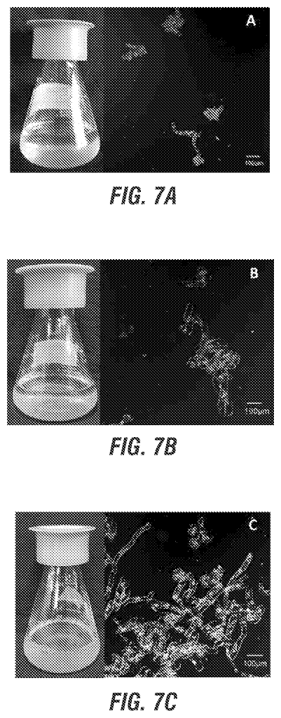

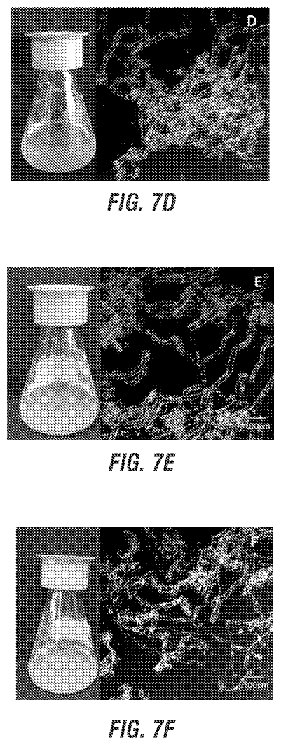

Phenotypically, the cell suspension culture became more homogeneous and vigorous after 1 mo of establishment. Initially, introducing callus into the liquid medium led to small aggregated calli, which settled on the bottom of the flask. After that, because of continuous shaking, the calli released cells (fine suspension culture) into the medium, which remained suspended. Microscopically, these cultures consisted of two dominant cell types: round/oval and elongated (FIG. 5). The round/oval cells tended to stay in small groups, whereas the elongated cells did not agglomerate (FIG. 6). Over time in culture, round/oval cells would transition to chains of multiple cell clusters (FIG. 7).

The growth of cell cultures was assessed using two parameters: density of cells (OD₆₀₀) and measurement of fresh weight (g). In order to determine cell density, turbidity was recorded using a microplate reader at an optical density at 600 nm, After vigorous mixing, 200 μl of cultures was transferred in a 96-well plate for measuring. Finally, for measuring fresh weight, cells were harvested using bottle-top filters under constant vacuum pressure. Cell weight was determined by subtracting the weight of wet filter paper from the weight of filter paper plus cells. Data were taken during a time course of 10 days to estimate growth characteristics.

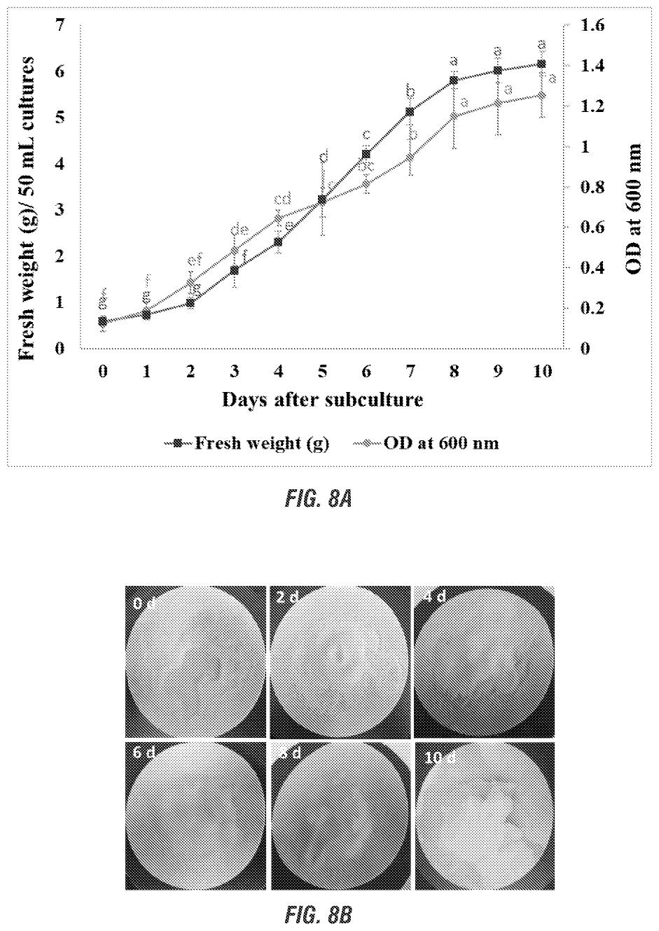

Based on cell culture fresh weight and turbidity measurements over time, growth rates could be characterized (FIG. 8A). There was little apparent variation in growth during the first 2 d of cultures. Over time, the fresh weight and cell culture turbidity gradually increased. At 8 d, fresh weight and turbidity peaked and eventually, after 8 d of culture there was negligible increase in cell growth. Visually, changes in cell concentration during culturing could be observed on filter paper (FIG. 8B). During active proliferation, the cultures were subcultured weekly to prevent overgrowth.

### Example 3: Protoplast Isolation from Cell Cultures, Stems, Leaves and Immature Cotyledons

Three different ages of cultures: 3, 4 and 5 d after subculturing were used for isolating protoplasts. Initially, 50 mL cultures were transferred in a 50 mL Falcon tube, allowed to settle for 1-h and the supernatant removed. Then 20 mL of fresh buffer solution (0.6 M mannitol, 10 mM 2-(N-morphilino) ethanesulfonic acid (MES); pH 5.7, 1 mM CaCl₂, 20 mM KCl, 0.1% bovine serine albumin (BSA) and 5 mM 2-mercaptoethanol) containing food-grade enzymes (Rohament CL 792.0 ECU, Rohapect 10 L 5040 ADJU, and Rohapect UF 0.039 ADJU) was filter-sterilized into the 50 mL Falcon tubes containing approximately 5 mL of PCV. The tube was then immediately placed horizontally in a shaking incubator at 24° C. and 90 rpm in the dark for 1.5 h.

After incubation, the solution was filtered through a 40 μm nylon mesh to remove large tissue fragments and centrifuged at 100×G for 3 min to pellet the protoplasts. The supernatant was removed, and the protoplast pellet was resuspended in 5 ml of washing solution (0.6 M mannitol, 4 mM MES; pH 5.7, 20 mM KCl). Total protoplast yield was quantified using a hemocytometer for 5 mL PCV. In order to separate debris, broken and intact protoplasts, 5 mL of 23% sucrose solution was carefully added into the bottom of the tube and centrifuged at 100×G for 3 min. After centrifugation, intact protoplasts were found at the interface of the two solutions and a cut pipette tip was used to carefully transfer the protoplast layer into new Falcon tubes. Protoplasts were then centrifuged under the same conditions to obtain a pellet; which was resuspended in W5 solution (154 mM NaCl, 125 mM CaCl₂), 5 mM KCl, 2 mM MES; pH 5.7). Protoplasts were observed under a microscope and quantified again using hemocytometer to determine the concentration of intact protoplasts. The resuspended solution was stored immediately after counting on ice prior to the transfection.

Stem and immature cotyledon experiments followed the same digestion procedure as cell suspension cultures. The enzyme and buffer solution was the same as cell suspension cultures. For stems, plants were grown in an environmental growth chamber with a 16/8 h photoperiod at 25° C. Stem tissues were harvested from 10-13-d-old plants. After collecting stems; the true leaf and cotyledons were removed from stems, and tissue was sliced into 0.1 cm thick pieces. Approximately 1-g of cut tissue was immediately, immersed into the enzyme solution to prevent drying. A vacuum was applied for 30 min to increase solution contact with tissue surfaces.

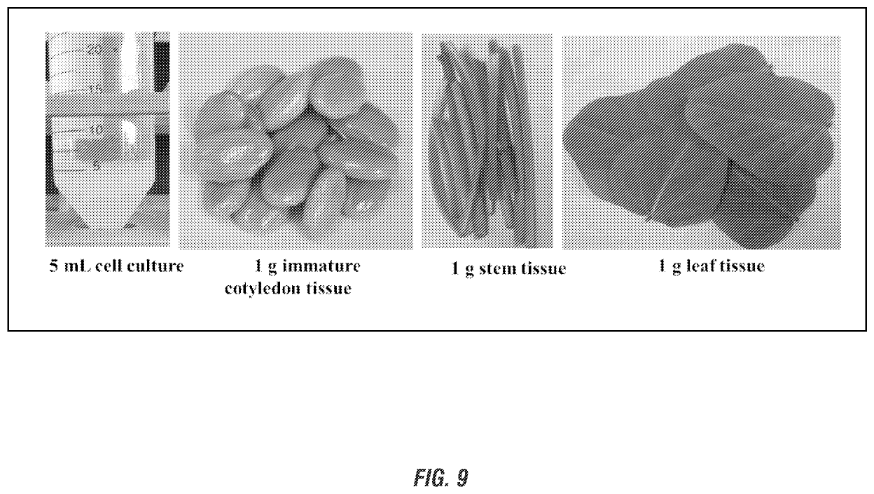

For immature cotyledons, plants were grown in a greenhouse under 16/8 h photoperiod at 26° C. Pods were harvested 30 d after flowering (FIG. 9). Immature cotyledons were collected after cross-sectioning of pods and sliced into 0.1 cm thick pieces. Approximately 1-g of sliced tissue was then transferred into enzyme solution without vacuum infiltration. Stem and immature cotyledon tissues were incubated with shaking at 30 rpm for 2 h at RT, followed by a wash procedure similar to that of the cell suspension cultures.

For leaf protoplast isolation, the isolation buffer used was the same as cell suspension cultures with different enzymes (0.5% cellulase, 0.5% macerozyme and 0.15% pectolyase Y23). For leaf tissue harvesting, plants were grown in a growth chamber under 16/8 h photoperiod at 25° C. for 7-10 d then plants were removed from growth chamber and incubated under dark conditions at RT for another 7 d. The dark-treated leaves were harvested (FIG. 9), sliced into 5 mm strips and immediately transferred into a petri dish containing enzyme solution. Leaf strips were vacuum infiltrated for 30 min then incubated at RT with gentle shaking at 30 rpm for 4-5 h. Both vacuum infiltration and incubation were performed in the dark. Following incubation, the petri dish was gently rotated manually for 5-10 min. During rotation, the buffer solution became green, indicating the release of protoplasts. The protoplast containing solution was then filtered through a 40 μm nylon mesh and washed.

### Example 4: Efficiency of Protoplast Isolation from Cell Cultures, Leaves, Stems and Immature Cotyledons

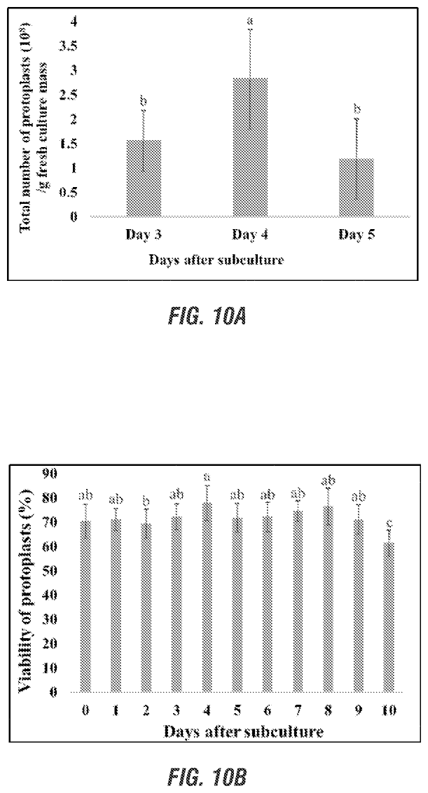

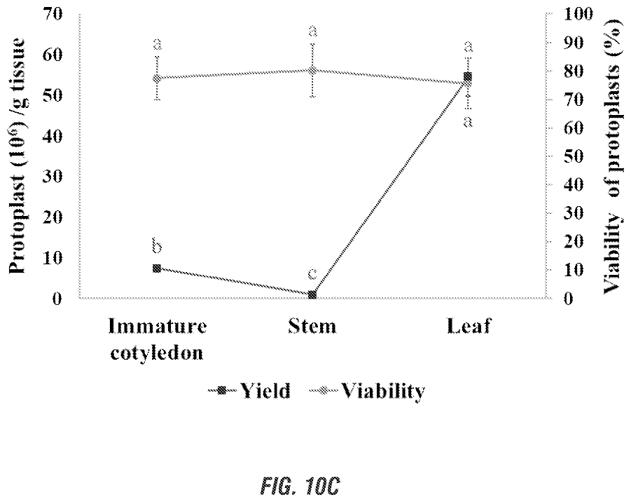

Isolation of protoplasts from the leaf-derived soybean cell suspension culture (LDSC), stems and immature cotyledons was achieved using low-cost food-grade enzymes (FIG. 9), We found that the LDSC produced the highest number of protoplasts 2.82±0.94×10⁸ per g fresh culture mass (FIG. 10A) while stems and immature cotyledons produced 9.8±0.30×10⁵ and 7.46±0.65×10⁶ protoplasts per g tissue, respectively (FIG. 10C). The yield of protoplasts from LDSC varied over time with 4-d-old cultures resulting in the highest yield (FIG. 10A). In leaf tissue, the food-grade enzymes proved ineffective at digestion and did not yield viable protoplasts. However, using a previously developed method and reagent-grade enzymes, 5.4±0.47×10⁷ protoplasts per g of leaf tissue were obtained (FIG. 10C).

The viability of protoplasts for all tissue sources was also determined by an Evans blue staining method. Evans blue dye was mixed with MMG solution (0.4 M mannitol, 15 mM MgCl₂ and 4 mM MFS pH 5.7) and added to the protoplasts to obtain a final concentration of 0.04%. After incubation at RT for 10 min, the number of viable protoplasts was determined using a hemocytometer. Viable protoplasts remained unstained from the blue dye while dead protoplasts were stained blue. The percent viability was calculated by dividing the number of live cells by the total number of cells counted. The maximum number of viable protoplasts was derived from 4-d-old cultures with 77±7.10% (FIG. 10B). After 8 days of culture, the viability decreased sharply, thus 4-d-old cultures were used for further experimentation. The viability of protoplasts was also measured in stems, immature cotyledons and leaves and ranged from 75-80%, (FIG. 10C).

### Example 5: PEG-Mediated Protoplast Transfection

Protoplasts were allowed to settle in the Falcon tube during 1 h incubation on ice. Then the supernatant was removed, and the pellet was resuspended at a concentration of 1×10⁵ mL⁻¹ using MMG solution. During transfection, plasmid DNA (10 μg) was placed into each of the transparent 14 mL Falcon round-bottom polystyrene tubes followed by 200 μl protoplast solution. Protoplasts and DNA were gently mixed with an equal volume of freshly prepared 40% PEG solution (4 g of PEG 4000, 3 mL of H₂O, 2.5 mL of 0.8M D-mannitol and 1 mL of 1 M CaCl₂). After gently shaking the transformation mixture by hand until homogenously mixed, it was incubated at RT for 10, 15 and 20 min. During incubation, the mixture was gently mixed every 5 min to prevent settling of protoplasts. After incubation, 1 mL W5 solution was added to terminate the reaction followed by centrifugation at 100×G for 3 min. The supernatant was then discarded and 200 μl WI solution was added (0.6 M of mannitol, 4 mM of KCl, 4 mM of MES, pH 5.7). After gentle mixing, the solution was transferred to a 96-well plate and incubated overnight at RT. Six biological replicates were performed for protoplast transformation from each source (leaf, cell culture, stem and immature cotyledon).

### Example 6: Optimization of PEG Incubation Time on Protoplast Transformation Efficiency

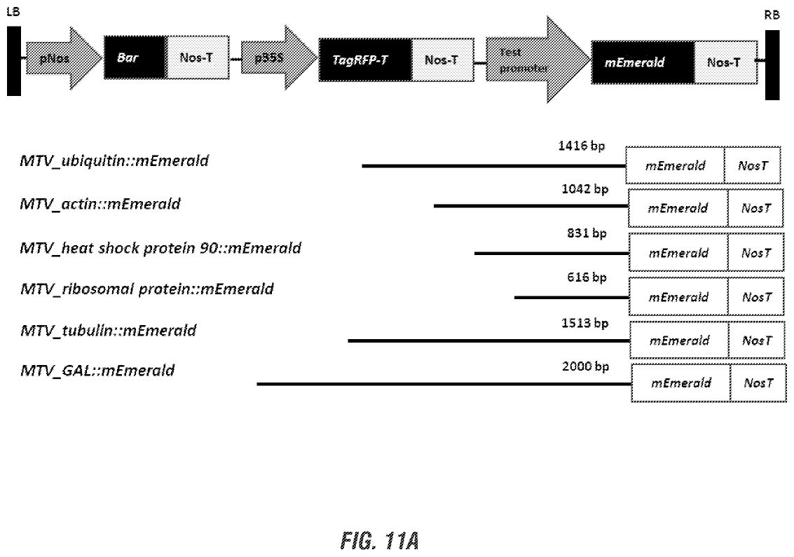

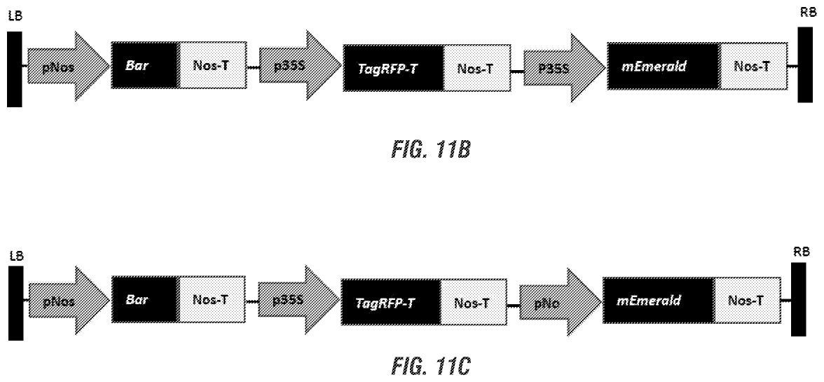

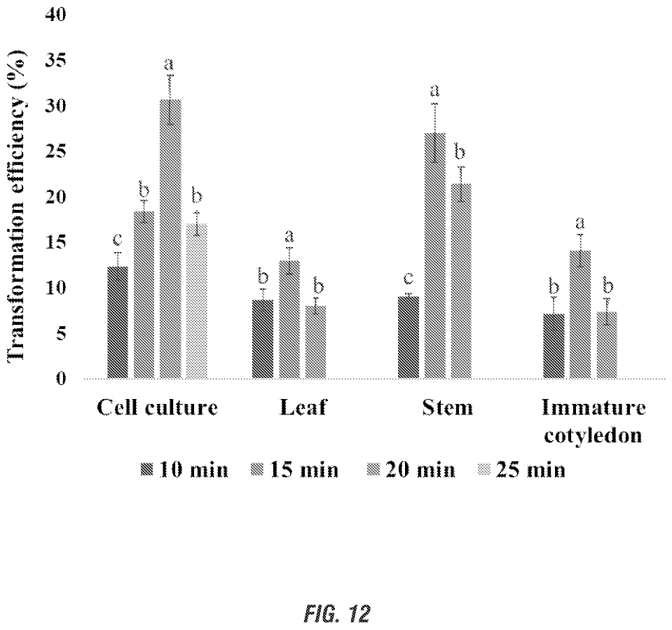

To further utilize the isolated protoplasts for functional analysis of promoters, we optimized the PEG incubation time for protoplasts from all sources. The binary vector (pB2GW7: 9983 bp) carrying the reporter gene GFP variant mEmerald (FIG. 11) was used to study the effect of PEG incubation time on soybean protoplast transformation efficiency. To optimize the PEG incubation duration for the four different protoplast sources, we examined the effect of 10, 15, 20- and 25-min transfection time (FIG. 12). Increasing transfection time from 10 min to 15 min resulted in an increase in average transformation efficiency in leaf (12.94±1.45%), stem (27.01±3.2%) and immature cotyledons (14.06±1.75%). When increased further from 15 min to 20 min, a decrease in transformation efficiency was observed (FIG. 12). This suggests that 15 min was an optimum transfection time for protoplasts from leaf, stem and immature cotyledon that resulted in maximum transformation efficiency. The transformation efficiency of LDSC-derived protoplasts reached at maximum at 20 min transfection time with PEG: 31.06±7.69% efficiency. Increasing transfection time from 20 to 25 min resulted in a decrease in transformation efficiency (FIG. 12).

### Example 7: Assessment of Promoter Directed Protoplast Transient Expression

Six soybean promoters were selected for testing the protoplast system based on prior characterization: ubiquitin, actin, heat-shock protein 90, ribosomal protein, tubulin, and GAL (α-galactosidase). Table 1 shows the Gene IDs for the genes and the respective sizes of their isolated promoters. ‘Williams 82’ genomic DNA was extracted using a routine method from 2-week-old leaves. Primers for amplifying promoter DNA were designed in Snapgene from the soybean genome (Phytozome database v12.1). Restriction sites were incorporated in the forward (EcoRI or AbsI or PacI) and reverse (NcoI) primers for directional cloning. Table 2 shows the list of primer sequences used for PCR amplification; restriction sites are underlined, and the appropriate restriction enzyme is indicated in parentheses.

TABLE 1

| Promoter | Size (bp) | Gene ID |
|---|---|---|
| Actin | 1042 | Glyma.19G147900 |
| Ribosomal protein | 616 | Glyma.09G094200 |
| Hsp90 | 831 | Glyma.08G332900 |
| Ubiquitin | 1416 | Glyma.20G141600 |
| Tubulin | 1513 | Glyma.05G207500 |
| GAL | 2000 | Glyma.03G137900 |

TABLE 2

| Promoter | Primer sequence (5′ to 3′) |
|---|---|
| CaMV 35S | F: ATACTTGAATTCTGAGACTTTTCAACAAAGGGTAATATCGGG (EcoRI) |
| | (SEQ ID NO: 1) |
| | R: ATACTTCCATGGTCAGCGTGTCCTCTCCAAATGAAAT (NcoI) |
| | (SEQ ID NO: 2) |
| Actin | F: CACCAAAGAATTCACTTTAACAGCAACACAATTTACAAT (EcoRI) |
| | (SEQ ID NO: 3) |
| | R: GGACGTCCATGGGGTTGTTTAAGGTAAAAGATGTTTGT (NcoI) |
| | (SEQ ID NO: 4) |
| Ribosomal | F: ATTACGGAATTCATCTACAAGTATAGGTTATTTGTCATGC (EcoRI) |
| protein | (SEQ ID NO: 5) |
| | R: AATCGCCCATGGGGTTGAGGCACTGTTTCAA (NcoI) |
| | (SEQ ID NO: 6) |
| Hsp90 | F: CGGAGGAATTCAAATAAATGGAAATCCACTCTAAAAAAA (EcoRI) |
| | (SEQ ID NO: 7) |
| | R: AATCGCCCATGGTGTCGATCTACGCGAG (NcoI) |
| | (SEQ ID NO: 8) |
| Ubiquitin | F: CACCTGGAATTCTCCTTAAGTTGCAGCATTTAACACATCTCCTC (EcoRI) |
| | (SEQ ID NO: 9) |
| | R: TGCCATCCATGGTACCTGTCGAGTCAACAATCACAGATAAATCAGAA (NcoI) |
| | (SEQ ID NO: 10) |
| Tubulin | F: CACCTCCCTCGAGGCTGTATGAAATGATATAATATATTCACA (AbsI) |
| | (SEQ ID NO: 11) |
| | R: CACCTCCCATGGTTTGAAGATAATTCAATTCAACT (NcoI) |
| | (SEQ ID NO: 12) |
| GAL | F: CACCTCTTAATTAATAGTTATTTGACTGGATTC (PacI) |
| | (SEQ ID NO: 13) |
| | R: CACCTCCCATGGTTTCGAACACTTCACCACTG (NcoI) |
| | (SEQ ID NO: 14) |

A dual-fluorescent protein gene (TagRFP and mEmerald) promoter screening vector, pMTV (FIG. 11), was specifically designed for this study. This vector contains dual plant selection, Nos:Bar for dicots and PvUbi1+3:Hygromycin for monocots, and UASrpg insulators in between each cassette to prevent cross-talk between promoters. In addition, pMTV contains right and left borders to enable Agrobacterium-mediated insertion into the genome. For this study, the reference promoter screening cassette was CaMV 35S::TagRFP orange fluorescent protein (OFP), while the test promoter screening cassette was_::mEmerald green fluorescent protein (GFP) reporter gene (FIG. 11), This design enabled green-to-red fluorescence ratios to be calculated for each of the test promoters to gauge promoter strength relative to the 35S promoter. To clone in the test promoters, a PCR amplicon for each promoter was purified, then purified products were digested using appropriate restriction enzymes (EcoRI and NcoI or AbsI and NcoI or PacI and NcoI). The digested product was ligated immediately upstream of the GFP according to the protocol (FIG. 11A). A 35S::GFP was used as a positive control (FIG. 11B) and a promoter-less construct was used as the negative control (FIG. 11C) for each transformation experiment. After cloning, the PCR amplicons from the complete test cassettes were sequence-verified.

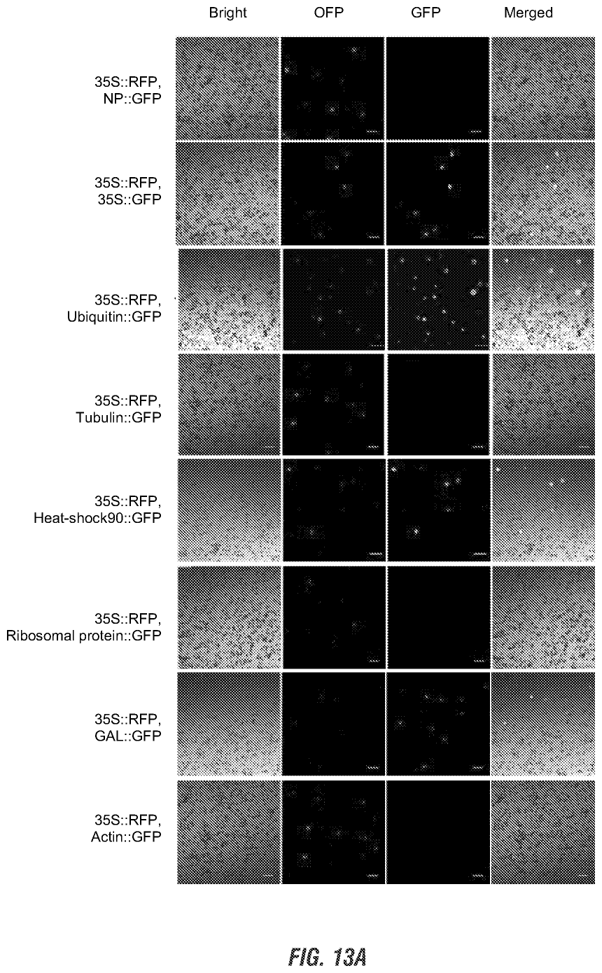

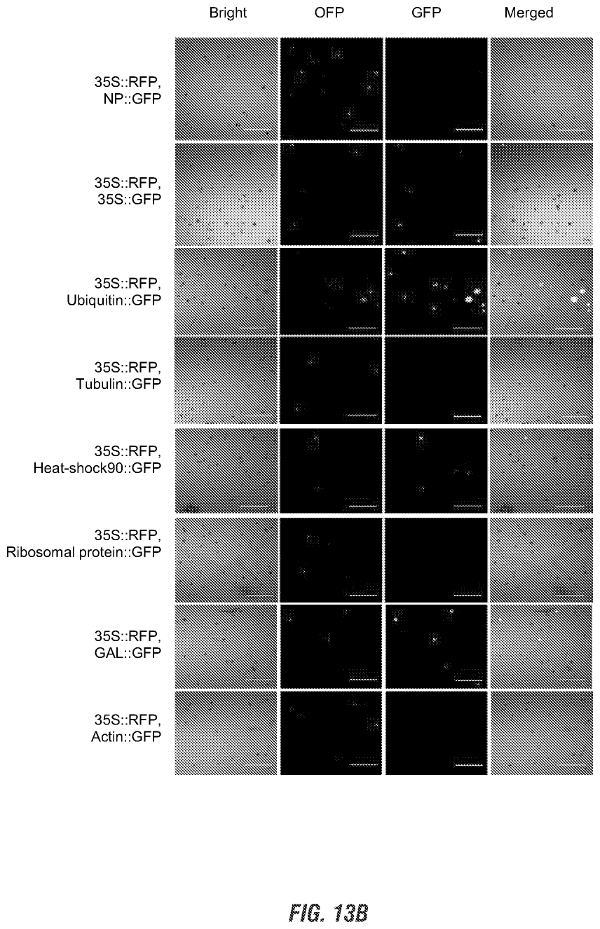

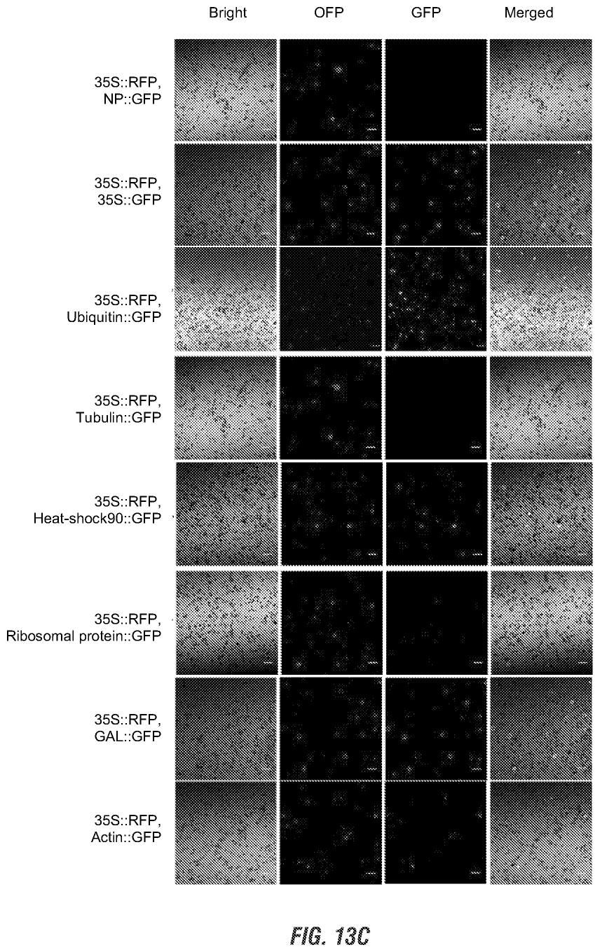

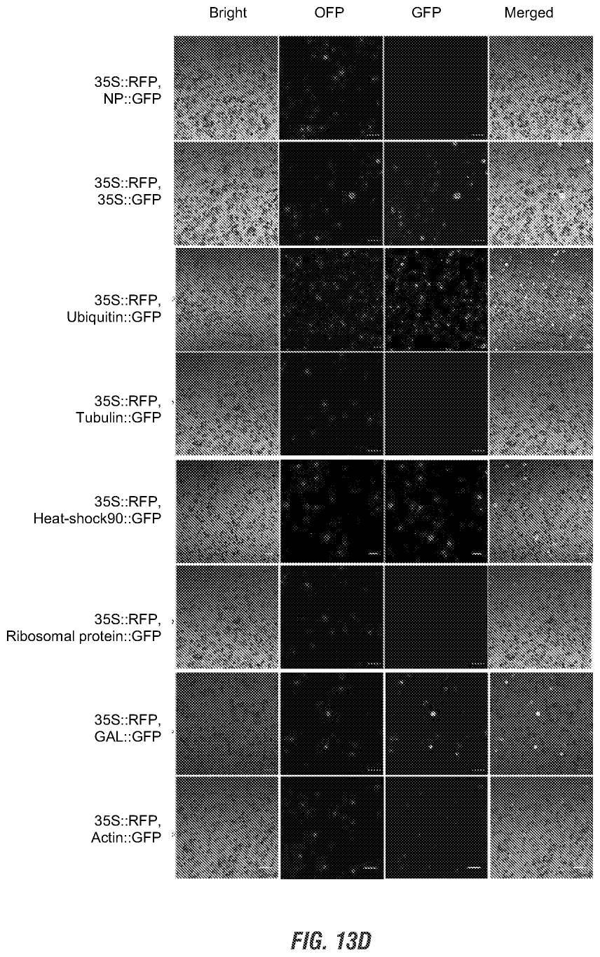

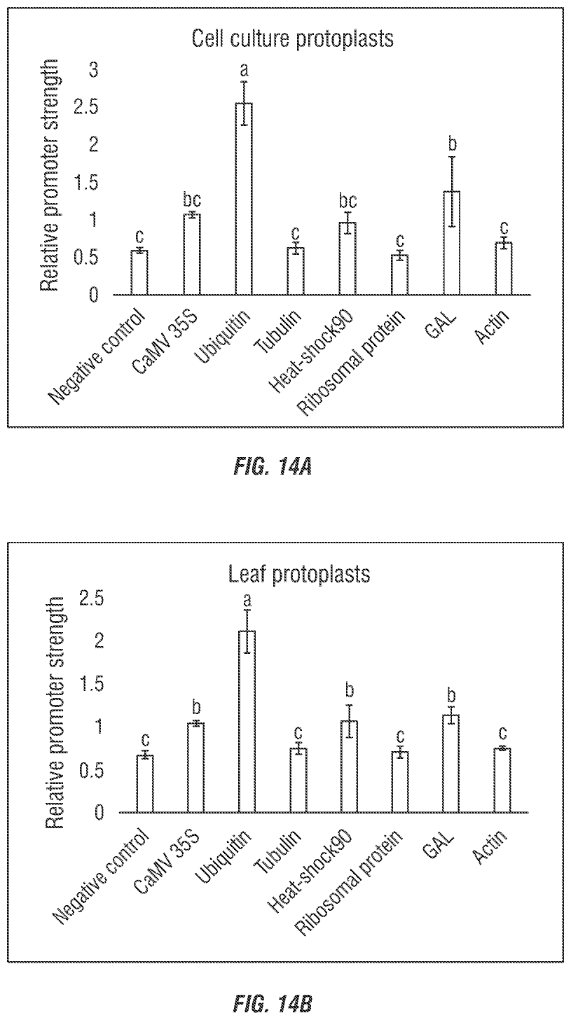

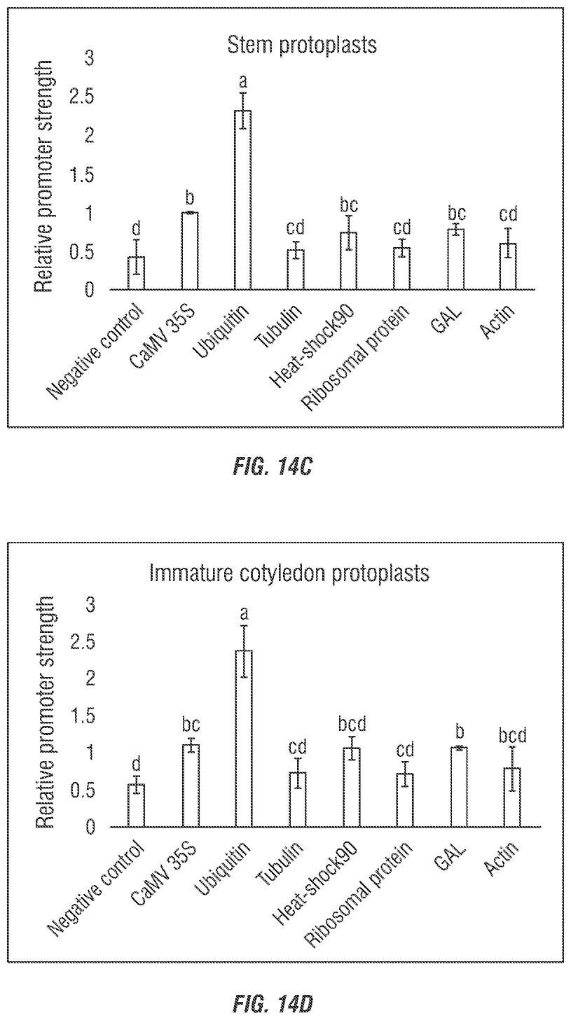

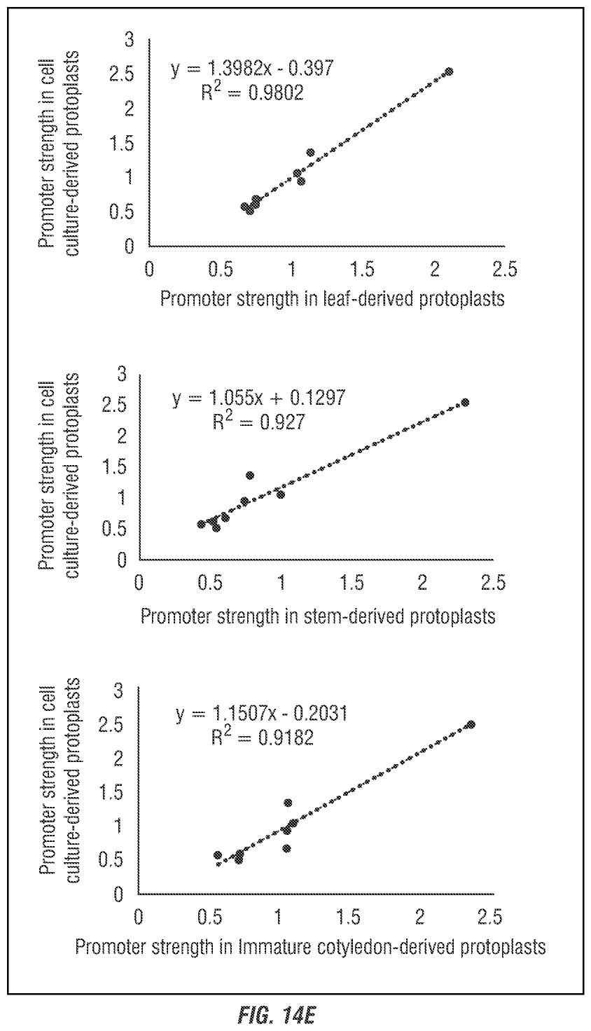

At 48 h post transfection, the ubiquitin promoter-directed expression in LDSC-derived protoplasts was very high, whereas expression was very low from promoters for tubulin, ribosomal protein and actin as assessed by microscopy (FIG. 13A). A moderate level of GFP expression was observed in protoplasts under the control of the 35S, Hsp90 and GAL promoters (FIG. 13A). In protoplasts derived from all sources, the 35S reference promoter drove OFP expression (FIG. 13A-D). Quantitative data was obtained from the ratio of GFP:OFP fluorescence in which data represent relative promoter:GFP strength to the internal 35S:OFP. The ubiquitin:35S promoter ratio for GFP expression was 2.54 for cell culture, 2.11 for leaf, 2.31 for stem and 2.36 for immature cotyledon (FIG. 14A-D). These results suggested that the ubiquitin promoter was nearly twice the strength of the 35S promoter. It was also apparent that the 35S, Hsp90 and GAL had similar promoter strength to drive GFP expression; for LDSC (1.05±0.08, 0.95±0.33 and 1.36±1.13), for leaf (1.04±0.03, 1.07±0.19 and 1.13±0.10), for stem (0.99±0.009, 0.74±0.22 and 0.78±0.08) and for immature cotyledon (1.03±0.09, 1.06±0.15 and 1.07±0.29) (FIG. 14A-D). While tubulin, ribosomal protein and actin promoters had a very low GIP expression in LDSC (0.61±0.18, 0.52±0.16 and 0.68±0.19), in leaf (0.75±0.06, 0.71±0.07 and 0.75±0.02), in stem (0.52±0.11, 0.54±0.11 and 0.60±0.12) and in immature cotyledon (0.73±0.20, 0.71±0.16 and 0.78±0.29) (FIG. 14A-D). To predict the association of promoter strength between LDSCs and other sources protoplasts, correlation analysis and simple linear regression were performed. We found that the promoter function in LDSC was strongly related with leaf (y=1.3982x−0.397, R²=0.9802), stem (y=1.055x+0.1297, R²=0.927) and immature cotyledon (y=1.1507x−0.2031, R²=0.9182) (FIG. 14E). We also found correlation coefficient for leaf and LDSC is 0.99, for stem and LDSC is 0.96 and for immature cotyledon and LDSC is 0.95.

### Example 8: Gene Expression Analysis of Soybean Tissues

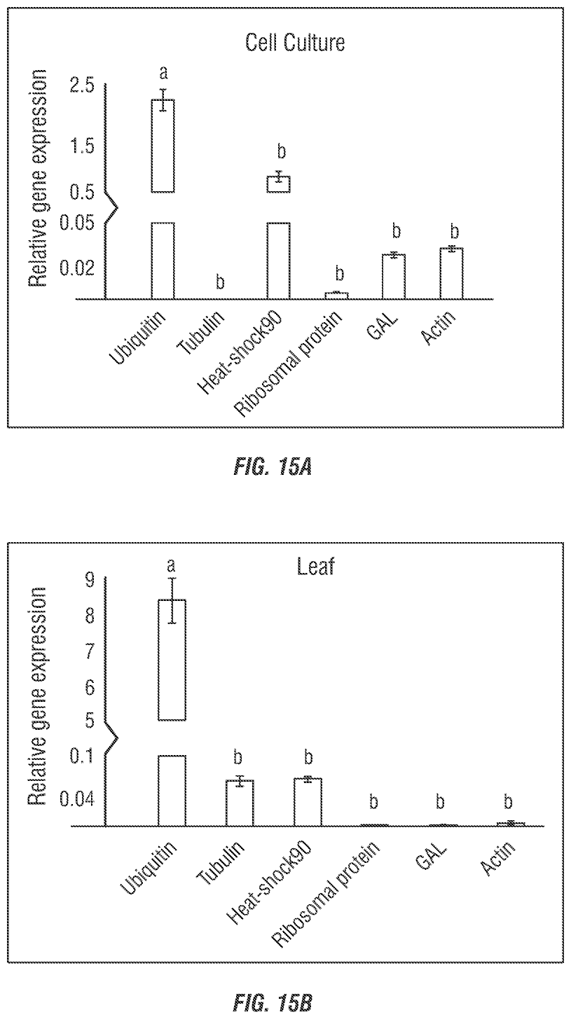

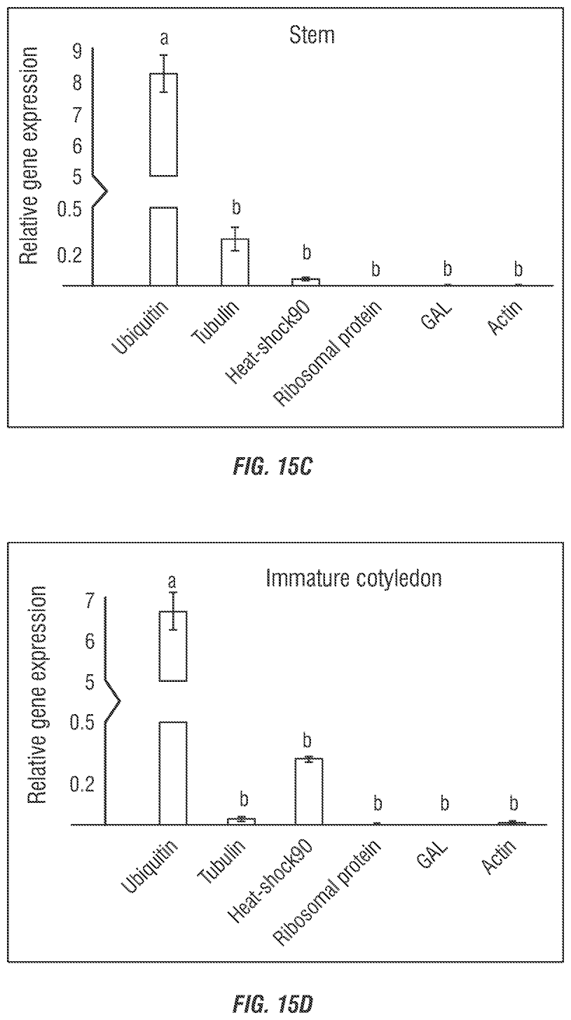

To test whether the promoter of interest driving endogenous gene expression provides a similar pattern, qRT-PCR was conducted in native tissues. Gene-specific primers corresponding to each promoter were designed; Table 3 shows the list of primer sequences used for GIRT-PCR amplification. We found the relative transcripts of ubiquitin ti was significantly higher than all other promoters/genes tested in each tissue source (FIG. 15A-D). There were no significant differences in relative transcript abundance except for ubiquitin in all tissues tested (FIG. 15A-D). We also tested for statistical significance when excluding ubiquitin gene expression to test whether any differences exist among the low expressed genes. The result showed significant gene expression differences; for cell culture (Hsp90 was different relative to other genes), for leaf (Hsp90 and tubulin were different relative to other genes), for stem (tubulin was different relative to other genes) and for immature cotyledon (Hsp90 was different relative to other genes). Table 4 shows the relative expression of the endogenous genes in soybean tissues. Comparisons were made among genes using mean±SD of gene expression which was normalized to soybean ubiquitin gene. Same superscript letters above mean value indicate no significant difference according to the ANOVA Fisher's LSD test (p<0.05).

TABLE 3

| Gene | Primer sequence (5′ to 3′) |
|---|---|
| GmUB13 (Ref) | F: GTGTAATGTTGGATGTGTTCCC (SEQ ID NO: 15) |
| | R: ACACAATTGAGTTCAACACAAACCG (SEQ ID NO: 16) |
| Actin11 (Ref) | F: ATCTTGACTGAGCGTGGTTATTCC (SEQ ID NO: 17) |
| | R: GCTGGTCCTGGCTGTCTCC (SEQ ID NO: 18) |
| Actin | F: GGCACCTCTTAATCCTAA (SEQ ID NO: 19) |
| | R: ATAGCGACATACATAGCA (SEQ ID NO: 20) |
| Ribosomal protein | F: AAGGACCATTATTGTAAGGA (SEQ ID NO: 21) |
| | R: ATTGAACCTCACTGTCTTCG (SEQ ID NO: 22) |
| Hsp90 | F: GAAGCCCATTTGGATGAGAA (SEQ ID NO: 23) |
| | R: GATAAAGACACGGCGGACAT (SEQ ID NO: 24) |
| Ubiquitin | F: ACTTGGTGTTGCGTCTTCGT (SEQ ID NO: 25) |
| | R: GCTTGCCAGCAAAAATCAG (SEQ ID NO: 26) |
| Tubulin | F: CAACCAAATTGGAGGCAAGT (SEQ ID NO: 27) |
| | R: AAGGACCAGAACGCAAGCTA (SEQ ID NO: 28) |
| GAL | F: AAGGGGTCTTGTGACTGGTG (SEQ ID NO: 29) |
| | R: TCCCACAAGTTCCCATTCTC (SEQ ID NO: 30) |

TABLE 4

| Gene | Cell culture | Leaf | Stem | Immature cotyledon |
|---|---|---|---|---|
| Tubulin | 2.3 × 10⁻⁰⁵ ± 8.96 × 10^{-06ab} | 0.063 ± 0.029^{a} | 0.298 ± 0.129^{a} | 0.026 ± 0.015^{b} |
| Hsp90 | 0.733 ± 0.063^{a} | 0.066 ± 0.007^{a} | 0.043 ± 0.018^{b} | 0.318 ± 0.024^{a} |
| Ribosomal | 0.005 ± 0.004^{b} | 0.001 ± 0.0003^{b} | 0.0007 ± 0.0002^{b} | 0.003 ± 0.0003^{b} |
| protein | | | | |
| GAL | 0.029 ± 0.003^{b} | 0.001 ± 0.0005^{b} | 0.002 ± 0.0005^{b} | 0.001 ± 0.0005^{b} |
| Actin | 0.034 ± 0.026^{b} | 0.004 ± 0.004^{b} | 0.005 ± 0.004^{b} | 0.011 ± 0.008^{b} |

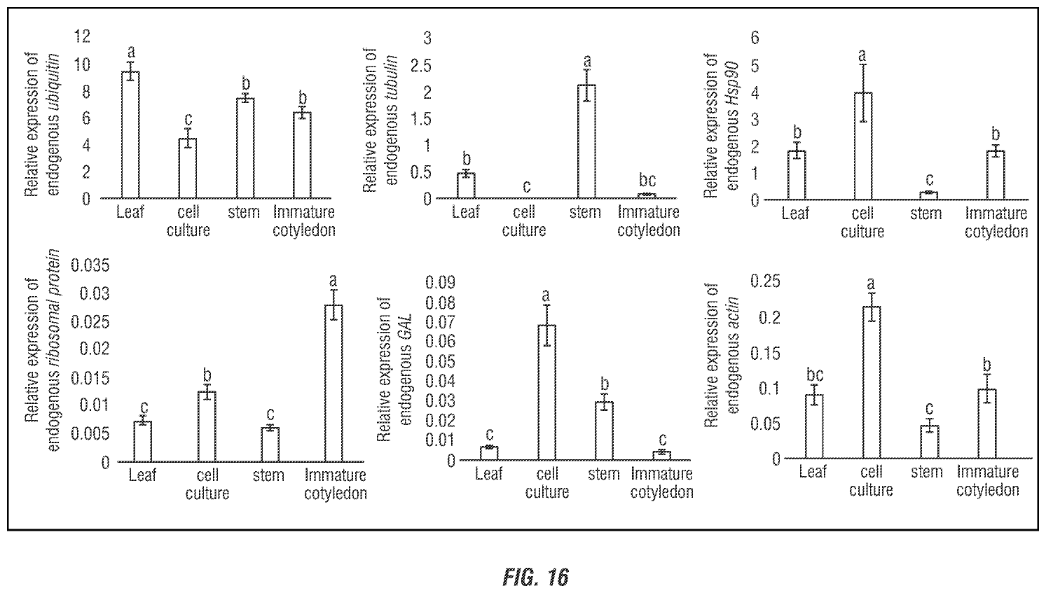

We also analyzed data using actin11 as the internal standard to determine relative transcripts abundance of endogenous genes in different tissue types. The overall conclusion was not changed from the previous analyses, yet these data provided insight from the additional housekeeping gene comparisons (FIG. 16), Ubiquitin transcripts were more abundant in leaf, stem and immature cotyledon compared to cell cultures, whereas the transcript levels of tubulin was minimal in all sources. Hsp90 showed intermediate transcripts abundance in cell culture relative to the leaf and immature cotyledon, whereas stems had very low transcripts. There were very low levels of transcripts abundance of ribosomal protein, GAL and actin from all sources.

### Example 9: Protoplast Expression Analysis after Automated Transfection

A protoplast transformation robot was used in feasibility-testing of the soybean system. The Lenaghan and Stewart (2019) protocol for tobacco BY-2 cell-based screening was used as the baseline for soybean cells to compare step efficiencies with the manual protoplast handling and transformation procedures described above. The robot was programmed to do the following procedure. Preloaded plasmid DNA (10 μL) and protoplasts (50 μL) in deep 96-well plates were placed into the nest. Preloaded 40% PEG, W5 and WI in column 1, 2 and 3 respectively in deep 96-well plate was placed in another nest. PEG solution (60 μL) was transferred into each well of deep 96-well plate containing protoplasts and plasmid DNA using a liquid handler. Then deep 96-well plate was moved to the plate shaker for mixing PEG and protoplasts. After that, protoplasts were allowed to incubate 20 min at RT and moved to large-volume liquid handler to add 240 μL of W5 to each well. The deep 96-well plate was removed from the robotic platform and centrifuged 100×G for 3 min. After centrifugation, the deep 96-well plate was placed again in the nest and the liquid handler was used to remove W5 solution and add 200 μL of WI solution. WI solution containing the protoplast was transferred into a 96-well screening plate.

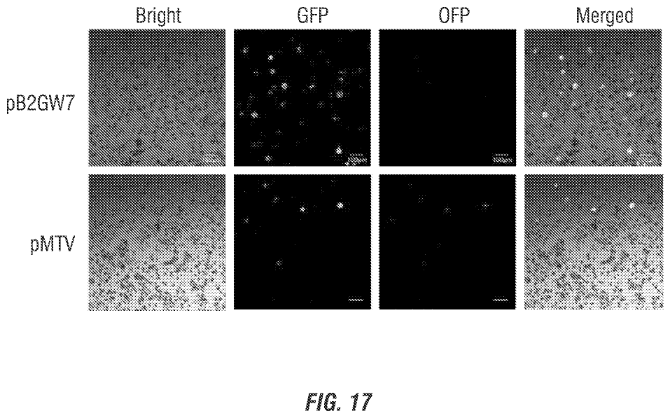

The LDSC-derived protoplast transformation protocol was successfully conducted using the robotic system. Automated transfected protoplasts were able to express reporter gene using two different plasmid DNA constructs (pB2GW7 and pMTV) (FIG. 17). Unfortunately; the transformation efficiency was low (4.32±0.64%) relative to the manual experiments (31.06±7.69%). The possible reason for low transformation could be the narrow pipette tip (50 mm); whereas for hand transformation we used wide pipette tip (70 mm). Another possible reason could be the problem of mixing PEG and protoplast. In our hands, we observed decreased protoplast transformation efficiency using 15 mL Falcon tubes instead of polystyrene tubes. Therefore, perhaps the mixing procedure in deep 96-well plate can cause low transformation rates. Optimization of robotic system protocols and components are needed to increase efficiency.

Automation of LDSC-derived protoplast transfection may be the most promising system to date for high-throughput promoter screening relevant to leaf traits. We identified at least two critical factors in the current protocol that need to be addressed to increase transformation frequency. A device that could handle even deeper well plates >0.06 ml and wide-bore pipette tips should improve protoplast handling and achieve increased transformation efficiency. In previous studies, automation of BY-2 protoplast transfection efficiency was shown ^{˜}2%; however this current study achieved better transfection (^{˜}4%) using LDSC-derived protoplasts. There may be other protocol modifications needed to increase the overall system efficiency for high-throughput screening. Nonetheless; we conclude the LDSC protoplasts are feasible to automate using the protoplast transformation robot.

### DEPOSITS

A deposit of the leaf-derived soybean cell suspension culture has been maintained at the University of Tennessee since prior to the filing date of this application. Access to these deposits will be available during the pendency of the application to the Commissioner of Patents and Trademarks and persons determined by the Commissioner to be entitled thereto upon request. Upon allowance of any claims in the application, the Applicant(s) will make available to the public without restriction a deposit of each of these cultures with the American Type Culture Collection (ATCC), Manassas, Va., 20110. The cells deposited with the ATCC will be taken from the same deposit maintained at the University of Tennessee as described above. Additionally, Applicant will meet all the requirements of 37 C.F.R. § 1.801-1.809, including providing an indication of the viability of the sample when the deposit is made. This deposit of the aforementioned cell lines will be maintained in the ATCC Depository, which is a public depository, for a period of 30 years, or 5 years after the most recent request, or for the enforceable life of the patent, whichever is longer, and will be replaced if it ever becomes nonviable during that period. Applicant will impose no restrictions on the availability of the deposited material from the ATCC; however, Applicant has no authority to waive any restrictions imposed by law on the transfer of biological material or its transportation in commerce.

1. A method of producing a plant cell suspension culture for protoplast isolation, the method comprising:
a) generating callus from leaf tissue;
b) transferring the callus to a liquid media to form a suspension of cells;
c) subculturing the suspension of cells under conditions sufficient to maintain the cells in a viable state; and
d) filtering the suspension of cells to remove large cell clusters.

2. The method of claim 1, wherein the generating callus from leaf tissue comprises placing the leaf tissue adaxial-side down in a callus induction media.

3. The method of claim 1, wherein the generating callus from leaf tissue comprises maintaining the leaf tissue under light for a time sufficient to generate callus.

4. The method of claim 3, wherein the time sufficient to generate callus is less than about three weeks.

5. The method of claim 2, wherein the callus induction media is supplemented with an auxin.

6. The method of claim 5, wherein the auxin is 2,4-dichlorophenoxyacetic acid.

7. The method of claim 2, wherein the callus induction media comprises from about 5 μM to about 40 μM 2,4-dichlorophenoxyacetic acid.

8. The method of claim 1, wherein the callus is divided into pieces prior to transferring the callus to the liquid media.

9. The method of claim 1, wherein the suspension of cells in step c) is maintained in the dark.

10. The method of claim 1, wherein the subculturing comprises collecting supernatant from the suspension of cells after allowing the suspension of cells to settle for a period of time.

11. The method of claim 1, wherein the filtering removes cell clusters larger than about 100 μm.

12. The method of claim 1, wherein the method further comprises cryopreserving the cell suspension culture.

13. The method of claim 1, wherein the plant is selected from soybean, potato, tomato, bean, pea, sunflower, maize, rice, barley, and wheat.

14. The method of claim 13, wherein the plant is soybean.

15. The method of claim 14, wherein the soybean plant is ‘Williams 82’.

16. The method of claim 1, wherein the method further comprises obtaining a protoplast from the cell suspension culture.

17. The method of claim 16, wherein the method further comprises introducing a nucleic acid into the protoplast.

18. The method of claim 17, wherein the nucleic acid comprises a gene, a promoter, a terminator, and/or an enhancer.

19. The method of claim 18, wherein the nucleic acid comprises a promoter.

20. The method of claim 19, wherein the promoter is selected from a ubiquitin promoter, an actin promoter, a heat-shock protein 90 promoter, a ribosomal protein promoter, a tubulin promoter, and an α-galactosidase promoter.

21. The method of claim 17, wherein the nucleic acid comprises a reporter gene.

22. The method of claim 17, wherein the introducing is by microinjection, electroporation, Agrobacterium-mediated transformation, polyethylene glycol (PEG)-mediated transformation, or microprojectile bombardment.

23. The method of claim 22, wherein the introducing is by PEG-mediated transformation.

24. The method of claim 17, wherein introducing the nucleic acid is automated.

25. A plant cell suspension culture produced according to the method of claim 1.

26. A protoplast obtained according to the method of claim 16.

27. The protoplast of claim 26, wherein the protoplast comprises an exogenous nucleic acid.

28. The protoplast of claim 26, wherein the protoplast is cryopreserved.

29. A method of obtaining a protoplast from a plant, the method comprising:
a) providing leaf tissue from the plant;
b) placing the leaf tissue adaxial-side down in callus induction media;
c) incubating under light for a time sufficient to generate callus;
d) dividing the callus into pieces;
e) transferring the callus pieces to a liquid media to form a suspension of cells;
f) subculturing the suspension of cells under conditions sufficient to maintain the cells in a viable state;
g) filtering the suspension of cells to remove large cell clusters;
h) recovering a cell from the liquid media; and
i) removing the cell wall from the cell with suitable enzymes to form a protoplast.

30. The method of claim 29, wherein the method further comprising introducing a nucleic acid into the protoplast.

31. A leaf-derived soybean cell suspension culture, wherein the cell suspension culture was deposited under ATCC Accession No. ______.

32. The cell suspension culture of claim 31, wherein the cell suspension culture is cryopreserved.

33. A protoplast produced from the cell suspension culture of claim 31.

34. The protoplast of claim 33, further comprising an exogenous nucleic acid.


SEQUENCE LISTING (30 sequences)

SEQ ID NO: 1 - DNA, 42 bp, Artificial Sequence
  Synthetic Construct
atacttgaattctgagacttttcaacaaagggtaatatcggg

SEQ ID NO: 2 - DNA, 37 bp, Artificial Sequence
  Synthetic Construct
atacttccatggtcagcgtgtcctctccaaatgaaat

SEQ ID NO: 3 - DNA, 39 bp, Artificial Sequence
  Synthetic Construct
caccaaagaattcactttaacagcaacacaatttacaat

SEQ ID NO: 4 - DNA, 38 bp, Artificial Sequence
  Synthetic Construct
ggacgtccatggggttgtttaaggtaaaagatgtttgt

SEQ ID NO: 5 - DNA, 40 bp, Artificial Sequence
  Synthetic Construct
attacggaattcatctacaagtataggttatttgtcatgc

SEQ ID NO: 6 - DNA, 31 bp, Artificial Sequence
  Synthetic Construct
aatcgcccatggggttgaggcactgtttcaa

SEQ ID NO: 7 - DNA, 39 bp, Artificial Sequence
  Synthetic Construct
cggaggaattcaaataaatggaaatccactctaaaaaaa

SEQ ID NO: 8 - DNA, 28 bp, Artificial Sequence
  Synthetic Construct
aatcgcccatggtgtcgatctacgcgag

SEQ ID NO: 9 - DNA, 44 bp, Artificial Sequence
  Synthetic Construct
cacctggaattctccttaagttgcagcatttaacacatctcctc

SEQ ID NO: 10 - DNA, 47 bp, Artificial Sequence
  Synthetic Construct
tgccatccatggtacctgtcgagtcaacaatcacagataaatcagaa

SEQ ID NO: 11 - DNA, 42 bp, Artificial Sequence
  Synthetic Construct
cacctccctcgaggctgtatgaaatgatataatatattcaca

SEQ ID NO: 12 - DNA, 35 bp, Artificial Sequence
  Synthetic Construct
cacctcccatggtttgaagataattcaattcaact

SEQ ID NO: 13 - DNA, 33 bp, Artificial Sequence
  Synthetic Construct
cacctcttaattaatagttatttgactggattc

SEQ ID NO: 14 - DNA, 32 bp, Artificial Sequence
  Synthetic Construct
cacctcccatggtttcgaacacttcaccactg

SEQ ID NO: 15 - DNA, 22 bp, Glycine max
gtgtaatgttggatgtgttccc

SEQ ID NO: 16 - DNA, 25 bp, Glycine max
acacaattgagttcaacacaaaccg

SEQ ID NO: 17 - DNA, 24 bp, Glycine max
atcttgactgagcgtggttattcc

SEQ ID NO: 18 - DNA, 19 bp, Glycine max
gctggtcctggctgtctcc

SEQ ID NO: 19 - DNA, 18 bp, Glycine max
ggcacctcttaatcctaa

SEQ ID NO: 20 - DNA, 18 bp, Glycine max
atagcgacatacatagca

SEQ ID NO: 21 - DNA, 20 bp, Glycine max
aaggaccattattgtaagga

SEQ ID NO: 22 - DNA, 20 bp, Glycine max
attgaacctcactgtcttcg

SEQ ID NO: 23 - DNA, 20 bp, Glycine max
gaagcccatttggatgagaa

SEQ ID NO: 24 - DNA, 20 bp, Glycine max
gataaagacacggcggacat

SEQ ID NO: 25 - DNA, 20 bp, Glycine max
acttggtgttgcgtcttcgt

SEQ ID NO: 26 - DNA, 19 bp, Glycine max
gcttgccagcaaaaatcag

SEQ ID NO: 27 - DNA, 20 bp, Glycine max
caaccaaattggaggcaagt

SEQ ID NO: 28 - DNA, 20 bp, Glycine max
aaggaccagaacgcaagcta

SEQ ID NO: 29 - DNA, 20 bp, Glycine max
aaggggtcttgtgactggtg

SEQ ID NO: 30 - DNA, 20 bp, Glycine max
tcccacaagttcccattctc